(19) United States
(12) Patent Application Publication
(10) Pub. No.: US 2017/0235879 A1
(43) Pub. Date: Aug. 17, 2017

(54) MOBILE TERMINAL AND METHOD FOR CONTROLLING THE SAME

(71) Applicant: LG ELECTRONICS INC., Seoul (KR)

(72) Inventors: Youngho SOHN, Seoul (KR); Jisoo PARK, Seoul (KR); Junjeong LEE, Seoul (KR); Woohyoung SHIN, Seoul (KR); Joohyung LEE, Seoul (KR); Junsu LEE, Seoul (KR)

(73) Assignee: LG Electronics Inc.

(21) Appl. No.: 15/282,646

(22) Filed: Sep. 30, 2016

(30) Foreign Application Priority Data
Feb. 16, 2016 (KR) 10-2016-0017948

(51) Int. Cl. G06F 19/00; G06F 3/0482; G06F 3/0488; H04N 5/232

(52) U.S. Cl. CPC G06F 19/321; G06F 19/322; G06F 19/3418; H04N 5/23229; G06F 3/0482; G06F 3/0488

(57) ABSTRACT

The present invention relates to a mobile terminal providing healthcare information and a method of controlling the same. According to an embodiment of the present invention, there is provided a mobile terminal including a camera; a display; and a controller configured to activate the camera to display a preview image on the display, to recognize a measurement value of a first health data measurement device, included in the preview image, and to provide healthcare information related to the recognized measurement value of first health data. Accordingly, the mobile terminal can interoperate with the heath data measurement device even when an additional interface for connecting the health data measurement device to the mobile terminal is not provided.

### CROSS-REFERENCE TO RELATED APPLICATION

Pursuant to 35 U.S.C §119(a), this application claims the benefit of earlier filing date and right of priority to Korean Patent Application No. 10-2016-0017948 filed on Feb. 16, 2016, the contents of which are hereby incorporated by reference in its entirety.

### BACKGROUND

### 1. Field

The present invention relates to a mobile terminal and a method for controlling the same.

### 2. Background

Terminals may be generally classified as mobile/portable terminals or stationary terminals according to their mobility. Mobile terminals may also be classified as handheld terminals or vehicle mounted terminals according to whether or not a user can directly carry the terminal.

Mobile terminals have become increasingly more functional. Examples of such functions include data and voice communications, capturing images and video via a camera, recording audio, playing music files via a speaker system, and displaying images and video on a display. Some mobile terminals include additional functionality which supports game playing, while other terminals are configured as multimedia players. More recently, mobile terminals have been configured to receive broadcast and multicast signals which permit viewing of content such as videos and television programs.

Efforts are ongoing to support and increase the functionality of mobile terminals. Such efforts include software and hardware improvements, as well as changes and improvements in the structural components.

Particularly, research on healthcare using a mobile terminal that a user carries all the time is underway.

### BRIEF DESCRIPTION OF THE DRAWINGS

The embodiments will be described in detail with reference to the following drawings in which like reference numerals refer to like elements wherein:

The present invention will become more fully understood from the detailed description given herein below and the accompanying drawings, which are given by illustration only, and thus are not limitative of the present invention, and wherein:

FIG. 1A is a block diagram of a mobile terminal in accordance with the present disclosure;

FIGS. 1B and 1C are conceptual views of one example of the mobile terminal, viewed from different directions;

FIGS. 2 and 3 are flowcharts illustrating control of the mobile terminal in accordance with the present disclosure;

FIG. 4 illustrates an environment for implementing a method for controlling the mobile terminal in accordance with the present disclosure;

FIGS. 5 to 9C are views illustrating a method for controlling the mobile terminal in accordance with the present disclosure;

FIGS. 10A to 14 are views illustrating a method for executing a camera preview function for photographing a health data measurement device in the mobile terminal in accordance with the present disclosure; and

FIGS. 15 to 22 are views illustrating a method for providing healthcare information through the mobile terminal in accordance with the present disclosure.

### DETAILED DESCRIPTION

Description will now be given in detail according to exemplary embodiments disclosed herein, with reference to the accompanying drawings. For the sake of brief description with reference to the drawings, the same or equivalent components may be provided with the same reference numbers, and description thereof will not be repeated. In general, a suffix such as “module” and “unit” may be used to refer to elements or components. Use of such a suffix herein is merely intended to facilitate description of the specification, and the suffix itself is not intended to give any special meaning or function. In the present disclosure, that which is well-known to one of ordinary skill in the relevant art has generally been omitted for the sake of brevity. The accompanying drawings are used to help easily understand various technical features and it should be understood that the embodiments presented herein are not limited by the accompanying drawings. As such, the present disclosure should be construed to extend to any alterations, equivalents and substitutes in addition to those which are particularly set out in the accompanying drawings.

It will be understood that although the terms first, second, etc. may be used herein to describe various elements, these elements should not be limited by these terms. These terms are generally only used to distinguish one element from another.

It will be understood that when an element is referred to as being “connected with” another element, the element can be connected with the other element or intervening elements may also be present. In contrast, when an element is referred to as being “directly connected with” another element, there are no intervening elements present.

A singular representation may include a plural representation unless it represents a definitely different meaning from the context. Terms such as “include” or “has” are used herein and should be understood that they are intended to indicate an existence of several components, functions or steps, disclosed in the specification, and it is also understood that greater or fewer components, functions, or steps may likewise be utilized.

Mobile terminals presented herein may be implemented using a variety of different types of terminals. Examples of such terminals include cellular phones, smart phones, user equipment, laptop computers, digital broadcast terminals, personal digital assistants (PDAs), portable multimedia players (PMPs), navigators, portable computers (PCs), slate PCs, tablet PCs, ultra books, wearable devices (for example, smart watches, smart glasses, head mounted displays (HMDs)), and the like.

By way of non-limiting example only, further description will be made with reference to particular types of mobile terminals. However, such teachings apply equally to other types of terminals, such as those types noted above. In addition, these teachings may also be applied to stationary terminals such as digital TV, desktop computers, and the like.

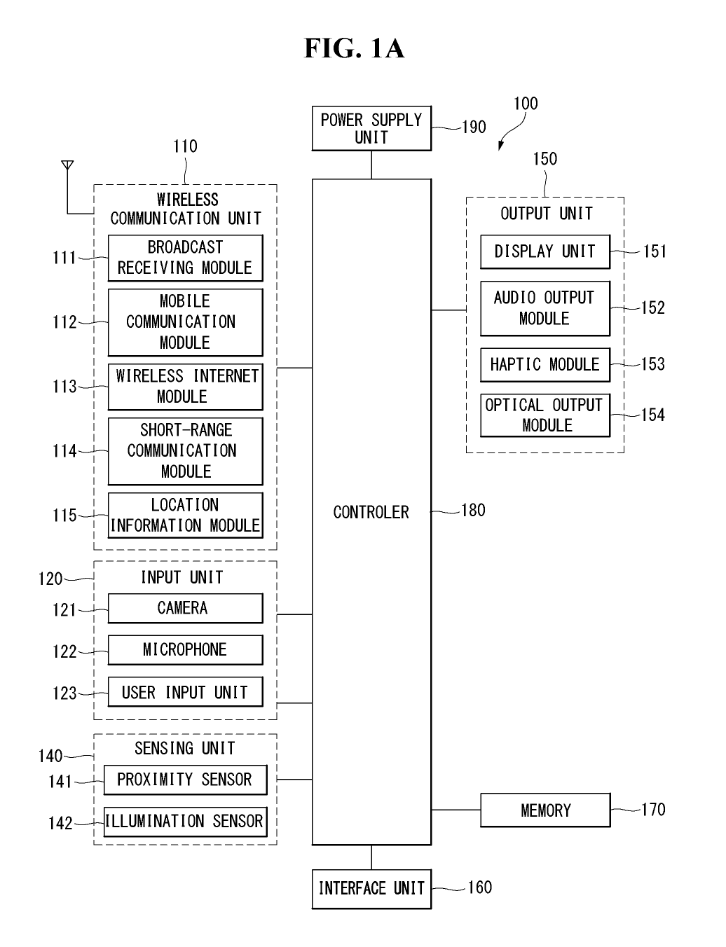

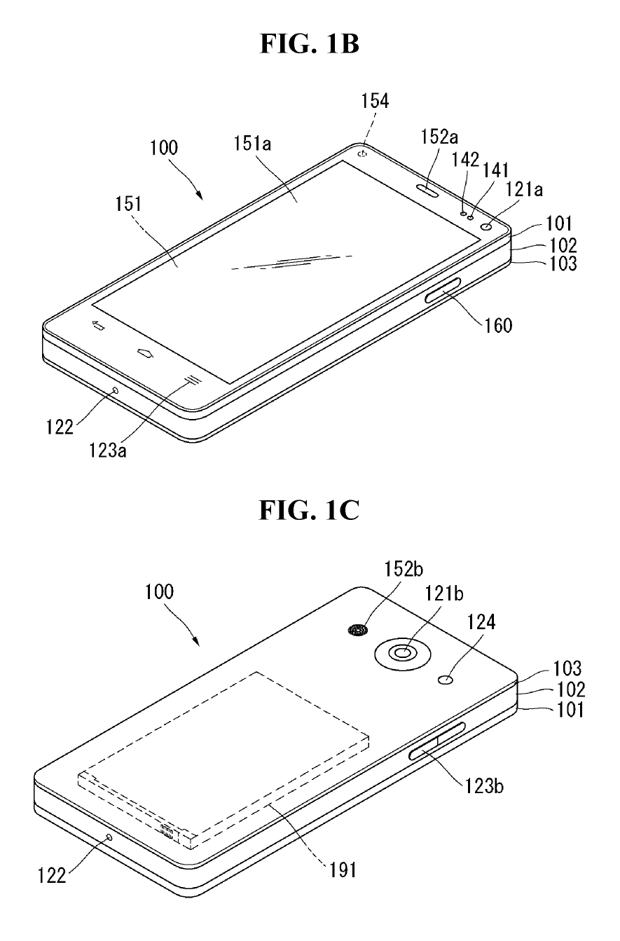

FIG. 1A is a block diagram of a mobile terminal in accordance with the present disclosure.

The mobile terminal 100 is shown having components such as a wireless communication unit 110, an input unit 120, a sensing unit 140, an output unit 150, an interface unit 160, a memory 170, a controller 180, and a power supply unit 190. It is understood that implementing all of the illustrated components is not a requirement, and that greater or fewer components may alternatively be implemented.

Referring now to FIG. 1A, the mobile terminal 100 is shown having wireless communication unit 110 configured with several commonly implemented components. For instance, the wireless communication unit 110 typically includes one or more components which permit wireless communication between the mobile terminal 100 and a wireless communication system or network within which the mobile terminal is located.

The wireless communication unit 110 typically includes one or more modules which permit communications such as wireless communications between the mobile terminal 100 and a wireless communication system, communications between the mobile terminal 100 and another mobile terminal, communications between the mobile terminal 100 and an external server. Further, the wireless communication unit 110 typically includes one or more modules which connect the mobile terminal 100 to one or more networks. To facilitate such communications, the wireless communication unit 110 includes one or more of a broadcast receiving module 111, a mobile communication module 112, a wireless Internet module 113, a short-range communication module 114, and a location information module 115.

The input unit 120 includes a camera 121 for obtaining images or video, a microphone 122, which is one type of audio input device for inputting an audio signal, and a user input unit 123 (for example, a touch key, a push key, a mechanical key, a soft key, and the like) for allowing a user to input information. Data (for example, audio, video, image, and the like) is obtained by the input unit 120 and may be analyzed and processed by controller 180 according to device parameters, user commands, and combinations thereof.

The sensing unit 140 is typically implemented using one or more sensors configured to sense internal information of the mobile terminal, the surrounding environment of the mobile terminal, user information, and the like. For example, in FIG. 1A, the sensing unit 140 is shown having a proximity sensor 141 and an illumination sensor 142.

If desired, the sensing unit 140 may alternatively or additionally include other types of sensors or devices, such as a touch sensor, an acceleration sensor, a magnetic sensor, a G-sensor, a gyroscope sensor, a motion sensor, an RGB sensor, an infrared (IR) sensor, a finger scan sensor, a ultrasonic sensor, an optical sensor (for example, camera 121), a microphone 122, a battery gauge, an environment sensor (for example, a barometer, a hygrometer, a thermometer, a radiation detection sensor, a thermal sensor, and a gas sensor, among others), and a chemical sensor (for example, an electronic nose, a health care sensor, a biometric sensor, and the like), to name a few. The mobile terminal 100 may be configured to utilize information obtained from sensing unit 140, and in particular, information obtained from one or more sensors of the sensing unit 140, and combinations thereof.

The output unit 150 is typically configured to output various types of information, such as audio, video, tactile output, and the like. The output unit 150 is shown having a display unit 151, an audio output module 152, a haptic module 153, and an optical output module 154.

The display unit 151 may have an inter-layered structure or an integrated structure with a touch sensor in order to facilitate a touch screen. The touch screen may provide an output interface between the mobile terminal 100 and a user, as well as function as the user input unit 123 which provides an input interface between the mobile terminal 100 and the user.

The interface unit 160 serves as an interface with various types of external devices that can be coupled to the mobile terminal 100. The interface unit 160, for example, may include any of wired or wireless ports, external power supply ports, wired or wireless data ports, memory card ports, ports for connecting a device having an identification module, audio input/output (I/O) ports, video I/O ports, earphone ports, and the like. In some cases, the mobile terminal 100 may perform assorted control functions associated with a connected external device, in response to the external device being connected to the interface unit 160.

The memory 170 is typically implemented to store data to support various functions or features of the mobile terminal 100. For instance, the memory 170 may be configured to store application programs executed in the mobile terminal 100, data or instructions for operations of the mobile terminal 100, and the like. Some of these application programs may be downloaded from an external server via wireless communication. Other application programs may be installed within the mobile terminal 100 at time of manufacturing or shipping, which is typically the case for basic functions of the mobile terminal 100 (for example, receiving a call, placing a call, receiving a message, sending a message, and the like). It is common for application programs to be stored in the memory 170, installed in the mobile terminal 100, and executed by the controller 180 to perform an operation (or function) for the mobile terminal 100.

The controller 180 typically functions to control overall operation of the mobile terminal 100, in addition to the operations associated with the application programs. The controller 180 may provide or process information or functions appropriate for a user by processing signals, data, information and the like, which are input or output by the various components depicted in FIG. 1A, or activating application programs stored in the memory 170. As one example, the controller 180 controls some or all of the components illustrated in FIGS. 1A-1C according to the execution of an application program that have been stored in the memory 170.

The power supply unit 190 can be configured to receive external power or provide internal power in order to supply appropriate power required for operating elements and components included in the mobile terminal 100. The power supply unit 190 may include a battery, and the battery may be configured to be embedded in the terminal body, or configured to be detachable from the terminal body.

Referring still to FIG. 1A, various components depicted in this figure will now be described in more detail. Regarding the wireless communication unit 110, the broadcast receiving module 111 is typically configured to receive a broadcast signal and/or broadcast associated information from an external broadcast managing entity via a broadcast channel. The broadcast channel may include a satellite channel, a terrestrial channel, or both. In some embodiments, two or more broadcast receiving modules 111 may be utilized to facilitate simultaneously receiving of two or more broadcast channels, or to support switching among broadcast channels.

The mobile communication module 112 can transmit and/or receive wireless signals to and from one or more network entities. Typical examples of a network entity include a base station, an external mobile terminal, a server, and the like. Such network entities form part of a mobile communication network, which is constructed according to technical standards or communication methods for mobile communications (for example, Global System for Mobile Communication (GSM), Code Division Multi Access (CDMA), CDMA2000 (Code Division Multi Access 2000), EV-DO (Enhanced Voice-Data Optimized or Enhanced Voice-Data Only), Wideband CDMA (WCDMA), High Speed Downlink Packet access (HSDPA), HSUPA (High Speed Uplink Packet Access), Long Term Evolution (LTE), LTE-A (Long Term Evolution-Advanced), and the like). Examples of wireless signals transmitted and/or received via the mobile communication module 112 include audio call signals, video (telephony) call signals, or various formats of data to support communication of text and multimedia messages.

The wireless Internet module 113 is configured to facilitate wireless Internet access. This module may be internally or externally coupled to the mobile terminal 100. The wireless Internet module 113 may transmit and/or receive wireless signals via communication networks according to wireless Internet technologies.

Examples of such wireless Internet access include Wireless LAN (WLAN), Wireless Fidelity (Wi-Fi), Wi-Fi Direct, Digital Living Network Alliance (DLNA), Wireless Broadband (WiBro), Worldwide Interoperability for Microwave Access (WiMAX), High Speed Downlink Packet Access (HSDPA), HSUPA (High Speed Uplink Packet Access), Long Term Evolution (LTE), LTE-A (Long Term Evolution-Advanced), and the like. The wireless Internet module 113 may transmit/receive data according to one or more of such wireless Internet technologies, and other Internet technologies as well.

In some embodiments, when the wireless Internet access is implemented according to, for example, WiBro, HSDPA, HSUPA, GSM, CDMA, WCDMA, LTE, LTE-A and the like, as part of a mobile communication network, the wireless Internet module 113 performs such wireless Internet access. As such, the Internet module 113 may cooperate with, or function as, the mobile communication module 112.

The short-range communication module 114 is configured to facilitate short-range communications. Suitable technologies for implementing such short-range communications include BLUETOOTH™, Radio Frequency IDentification (RFID), Infrared Data Association (IrDA), Ultra-WideBand (UWB), ZigBee, Near Field Communication (NFC), Wireless-Fidelity (Wi-Fi), Wi-Fi Direct, Wireless USB (Wireless Universal Serial Bus), and the like. The short-range communication module 114 in general supports wireless communications between the mobile terminal 100 and a wireless communication system, communications between the mobile terminal 100 and another mobile terminal 100, or communications between the mobile terminal and a network where another mobile terminal 100 (or an external server) is located, via wireless area networks. One example of the wireless area networks is a wireless personal area networks.

In some embodiments, another mobile terminal (which may be configured similarly to mobile terminal 100) may be a wearable device, for example, a smart watch, a smart glass or a head mounted display (HMD), which is able to exchange data with the mobile terminal 100 (or otherwise cooperate with the mobile terminal 100). The short-range communication module 114 may sense or recognize the wearable device, and permit communication between the wearable device and the mobile terminal 100. In addition, when the sensed wearable device is a device which is authenticated to communicate with the mobile terminal 100, the controller 180, for example, may cause transmission of data processed in the mobile terminal 100 to the wearable device via the short-range communication module 114. Hence, a user of the wearable device may use the data processed in the mobile terminal 100 on the wearable device. For example, when a call is received in the mobile terminal 100, the user may answer the call using the wearable device. Also, when a message is received in the mobile terminal 100, the user can check the received message using the wearable device.

The location information module 115 is generally configured to detect, calculate, derive or otherwise identify a position of the mobile terminal. As an example, the location information module 115 includes a Global Position System (GPS) module, a Wi-Fi module, or both. If desired, the location information module 115 may alternatively or additionally function with any of the other modules of the wireless communication unit 110 to obtain data related to the position of the mobile terminal.

As one example, when the mobile terminal uses a GPS module, a position of the mobile terminal may be acquired using a signal sent from a GPS satellite. As another example, when the mobile terminal uses the Wi-Fi module, a position of the mobile terminal can be acquired based on information related to a wireless access point (AP) which transmits or receives a wireless signal to or from the Wi-Fi module.

The input unit 120 may be configured to permit various types of input to the mobile terminal 120. Examples of such input include audio, image, video, data, and user input. Image and video input is often obtained using one or more cameras 121. Such cameras 121 may process image frames of still pictures or video obtained by image sensors in a video or image capture mode. The processed image frames can be displayed on the display unit 151 or stored in memory 170. In some cases, the cameras 121 may be arranged in a matrix configuration to permit a plurality of images having various angles or focal points to be input to the mobile terminal 100. As another example, the cameras 121 may be located in a stereoscopic arrangement to acquire left and right images for implementing a stereoscopic image.

The microphone 122 is generally implemented to permit audio input to the mobile terminal 100. The audio input can be processed in various manners according to a function being executed in the mobile terminal 100. If desired, the microphone 122 may include assorted noise removing algorithms to remove unwanted noise generated in the course of receiving the external audio.

The user input unit 123 is a component that permits input by a user. Such user input may enable the controller 180 to control operation of the mobile terminal 100. The user input unit 123 may include one or more of a mechanical input element (for example, a key, a button located on a front and/or rear surface or a side surface of the mobile terminal 100, a dome switch, a jog wheel, a jog switch, and the like), or a touch-sensitive input, among others. As one example, the touch-sensitive input may be a virtual key or a soft key, which is displayed on a touch screen through software processing, or a touch key which is located on the mobile terminal at a location that is other than the touch screen. On the other hand, the virtual key or the visual key may be displayed on the touch screen in various shapes, for example, graphic, text, icon, video, or a combination thereof.

The sensing unit 140 is generally configured to sense one or more of internal information of the mobile terminal, surrounding environment information of the mobile terminal, user information, or the like. The controller 180 generally cooperates with the sending unit 140 to control operation of the mobile terminal 100 or execute data processing, a function or an operation associated with an application program installed in the mobile terminal based on the sensing provided by the sensing unit 140. The sensing unit 140 may be implemented using any of a variety of sensors, some of which will now be described in more detail.

The proximity sensor 141 may include a sensor to sense presence or absence of an object approaching a surface, or an object located near a surface, by using an electromagnetic field, infrared rays, or the like without a mechanical contact. The proximity sensor 141 may be arranged at an inner region of the mobile terminal covered by the touch screen, or near the touch screen.

The proximity sensor 141, for example, may include any of a transmissive type photoelectric sensor, a direct reflective type photoelectric sensor, a mirror reflective type photoelectric sensor, a high-frequency oscillation proximity sensor, a capacitance type proximity sensor, a magnetic type proximity sensor, an infrared rays proximity sensor, and the like. When the touch screen is implemented as a capacitance type, the proximity sensor 141 can sense proximity of a pointer relative to the touch screen by changes of an electromagnetic field, which is responsive to an approach of an object with conductivity. In this case, the touch screen (touch sensor) may also be categorized as a proximity sensor.

The term “proximity touch” will often be referred to herein to denote the scenario in which a pointer is positioned to be proximate to the touch screen without contacting the touch screen. The term “contact touch” will often be referred to herein to denote the scenario in which a pointer makes physical contact with the touch screen. For the position corresponding to the proximity touch of the pointer relative to the touch screen, such position will correspond to a position where the pointer is perpendicular to the touch screen. The proximity sensor 141 may sense proximity touch, and proximity touch patterns (for example, distance, direction, speed, time, position, moving status, and the like).

In general, controller 180 processes data corresponding to proximity touches and proximity touch patterns sensed by the proximity sensor 141, and cause output of visual information on the touch screen. In addition, the controller 180 can control the mobile terminal 100 to execute different operations or process different data according to whether a touch with respect to a point on the touch screen is either a proximity touch or a contact touch.

A touch sensor can sense a touch applied to the touch screen, such as display unit 151, using any of a variety of touch methods. Examples of such touch methods include a resistive type, a capacitive type, an infrared type, and a magnetic field type, among others.

As one example, the touch sensor may be configured to convert changes of pressure applied to a specific part of the display unit 151, or convert capacitance occurring at a specific part of the display unit 151, into electric input signals. The touch sensor may also be configured to sense not only a touched position and a touched area, but also touch pressure and/or touch capacitance. A touch object is generally used to apply a touch input to the touch sensor. Examples of typical touch objects include a finger, a touch pen, a stylus pen, a pointer, or the like.

When a touch input is sensed by a touch sensor, corresponding signals may be transmitted to a touch controller. The touch controller may process the received signals, and then transmit corresponding data to the controller 180. Accordingly, the controller 180 may sense which region of the display unit 151 has been touched. Here, the touch controller may be a component separate from the controller 180, the controller 180, and combinations thereof.

In some embodiments, the controller 180 may execute the same or different controls according to a type of touch object that touches the touch screen or a touch key provided in addition to the touch screen. Whether to execute the same or different control according to the object which provides a touch input may be decided based on a current operating state of the mobile terminal 100 or a currently executed application program, for example.

The touch sensor and the proximity sensor may be implemented individually, or in combination, to sense various types of touches. Such touches includes a short (or tap) touch, a long touch, a multi-touch, a drag touch, a flick touch, a pinch-in touch, a pinch-out touch, a swipe touch, a hovering touch, and the like.

If desired, an ultrasonic sensor may be implemented to recognize position information relating to a touch object using ultrasonic waves. The controller 180, for example, may calculate a position of a wave generation source based on information sensed by an illumination sensor and a plurality of ultrasonic sensors. Since light is much faster than ultrasonic waves, the time for which the light reaches the optical sensor is much shorter than the time for which the ultrasonic wave reaches the ultrasonic sensor. The position of the wave generation source may be calculated using this fact. For instance, the position of the wave generation source may be calculated using the time difference from the time that the ultrasonic wave reaches the sensor based on the light as a reference signal.

The camera 121 typically includes at least one a camera sensor (CCD, CMOS etc.), a photo sensor (or image sensors), and a laser sensor.

Implementing the camera 121 with a laser sensor may allow detection of a touch of a physical object with respect to a 3D stereoscopic image. The photo sensor may be laminated on, or overlapped with, the display device. The photo sensor may be configured to scan movement of the physical object in proximity to the touch screen. In more detail, the photo sensor may include photo diodes and transistors at rows and columns to scan content received at the photo sensor using an electrical signal which changes according to the quantity of applied light. Namely, the photo sensor may calculate the coordinates of the physical object according to variation of light to thus obtain position information of the physical object.

The display unit 151 is generally configured to output information processed in the mobile terminal 100. For example, the display unit 151 may display execution screen information of an application program executing at the mobile terminal 100 or user interface (UI) and graphic user interface (GUI) information in response to the execution screen information.

In some embodiments, the display unit 151 may be implemented as a stereoscopic display unit for displaying stereoscopic images. A typical stereoscopic display unit may employ a stereoscopic display scheme such as a stereoscopic scheme (a glass scheme), an auto-stereoscopic scheme (glassless scheme), a projection scheme (holographic scheme), or the like.

The audio output module 152 is generally configured to output audio data. Such audio data may be obtained from any of a number of different sources, such that the audio data may be received from the wireless communication unit 110 or may have been stored in the memory 170. The audio data may be output during modes such as a signal reception mode, a call mode, a record mode, a voice recognition mode, a broadcast reception mode, and the like. The audio output module 152 can provide audible output related to a particular function (e.g., a call signal reception sound, a message reception sound, etc.) performed by the mobile terminal 100. The audio output module 152 may also be implemented as a receiver, a speaker, a buzzer, or the like.

A haptic module 153 can be configured to generate various tactile effects that a user feels, perceive, or otherwise experience. A typical example of a tactile effect generated by the haptic module 153 is vibration. The strength, pattern and the like of the vibration generated by the haptic module 153 can be controlled by user selection or setting by the controller. For example, the haptic module 153 may output different vibrations in a combining manner or a sequential manner.

Besides vibration, the haptic module 153 can generate various other tactile effects, including an effect by stimulation such as a pin arrangement vertically moving to contact skin, a spray force or suction force of air through a jet orifice or a suction opening, a touch to the skin, a contact of an electrode, electrostatic force, an effect by reproducing the sense of cold and warmth using an element that can absorb or generate heat, and the like.

The haptic module 153 can also be implemented to allow the user to feel a tactile effect through a muscle sensation such as the user's fingers or arm, as well as transferring the tactile effect through direct contact. Two or more haptic modules 153 may be provided according to the particular configuration of the mobile terminal 100.

An optical output module 154 can output a signal for indicating an event generation using light of a light source. Examples of events generated in the mobile terminal 100 may include message reception, call signal reception, a missed call, an alarm, a schedule notice, an email reception, information reception through an application, and the like.

A signal output by the optical output module 154 may be implemented in such a manner that the mobile terminal emits monochromatic light or light with a plurality of colors. The signal output may be terminated as the mobile terminal senses that a user has checked the generated event, for example.

The interface unit 160 serves as an interface for external devices to be connected with the mobile terminal 100. For example, the interface unit 160 can receive data transmitted from an external device, receive power to transfer to elements and components within the mobile terminal 100, or transmit internal data of the mobile terminal 100 to such external device. The interface unit 160 may include wired or wireless headset ports, external power supply ports, wired or wireless data ports, memory card ports, ports for connecting a device having an identification module, audio input/output (I/O) ports, video I/O ports, earphone ports, or the like.

The identification module may be a chip that stores various information for authenticating authority of using the mobile terminal 100 and may include a user identity module (UIM), a subscriber identity module (SIM), a universal subscriber identity module (USIM), and the like. In addition, the device having the identification module (also referred to herein as an “identifying device”) may take the form of a smart card. Accordingly, the identifying device can be connected with the terminal 100 via the interface unit 160.

When the mobile terminal 100 is connected with an external cradle, the interface unit 160 can serve as a passage to allow power from the cradle to be supplied to the mobile terminal 100 or may serve as a passage to allow various command signals input by the user from the cradle to be transferred to the mobile terminal there through. Various command signals or power input from the cradle may operate as signals for recognizing that the mobile terminal is properly mounted on the cradle.

The memory 170 can store programs to support operations of the controller 180 and store input/output data (for example, phonebook, messages, still images, videos, etc.). The memory 170 may store data related to various patterns of vibrations and audio which are output in response to touch inputs on the touch screen.

The memory 170 may include one or more types of storage mediums including a Flash memory, a hard disk, a solid state disk, a silicon disk, a multimedia card micro type, a card-type memory (e.g., SD or DX memory, etc.), a Random Access Memory (RAM), a Static Random Access Memory (SRAM), a Read-Only Memory (ROM), an Electrically Erasable Programmable Read-Only Memory (EEPROM), a Programmable Read-Only memory (PROM), a magnetic memory, a magnetic disk, an optical disk, and the like. The mobile terminal 100 may also be operated in relation to a network storage device that performs the storage function of the memory 170 over a network, such as the Internet.

The controller 180 may typically control the general operations of the mobile terminal 100. For example, the controller 180 may set or release a lock state for restricting a user from inputting a control command with respect to applications when a status of the mobile terminal meets a preset condition.

The controller 180 can also perform the controlling and processing associated with voice calls, data communications, video calls, and the like, or perform pattern recognition processing to recognize a handwriting input or a picture drawing input performed on the touch screen as characters or images, respectively. In addition, the controller 180 can control one or a combination of those components in order to implement various exemplary embodiments disclosed herein.

The power supply unit 190 receives external power or provides internal power and supply the appropriate power required for operating respective elements and components included in the mobile terminal 100. The power supply unit 190 may include a battery, which is typically rechargeable or be detachably coupled to the terminal body for charging.

The power supply unit 190 may include a connection port. The connection port may be configured as one example of the interface unit 160 to which an external charger for supplying power to recharge the battery is electrically connected.

As another example, the power supply unit 190 may be configured to recharge the battery in a wireless manner without use of the connection port. In this example, the power supply unit 190 can receive power, transferred from an external wireless power transmitter, using at least one of an inductive coupling method which is based on magnetic induction or a magnetic resonance coupling method which is based on electromagnetic resonance.

Various embodiments described herein may be implemented in a computer-readable medium, a machine-readable medium, or similar medium using, for example, software, hardware, or any combination thereof.

Referring now to FIGS. 1B and 1C, the mobile terminal 100 is described with reference to a bar-type terminal body. However, the mobile terminal 100 may alternatively be implemented in any of a variety of different configurations. Examples of such configurations include watch-type, clip-type, glasses-type, or as a folder-type, flip-type, slide-type, swing-type, and swivel-type in which two and more bodies are combined with each other in a relatively movable manner, and combinations thereof. Discussion herein will often relate to a particular type of mobile terminal (for example, bar-type, watch-type, glasses-type, and the like). However, such teachings with regard to a particular type of mobile terminal will generally apply to other types of mobile terminals as well.

The mobile terminal 100 will generally include a case (for example, frame, housing, cover, and the like) forming the appearance of the terminal. In this embodiment, the case is formed using a front case 101 and a rear case 102. Various electronic components are incorporated into a space formed between the front case 101 and the rear case 102. At least one middle case may be additionally positioned between the front case 101 and the rear case 102.

The display unit 151 is shown located on the front side of the terminal body to output information. As illustrated, a window 151a of the display unit 151 may be mounted to the front case 101 to form the front surface of the terminal body together with the front case 101.

In some embodiments, electronic components may also be mounted to the rear case 102. Examples of such electronic components include a detachable battery 191, an identification module, a memory card, and the like. Rear cover 103 is shown covering the electronic components, and this cover may be detachably coupled to the rear case 102. Therefore, when the rear cover 103 is detached from the rear case 102, the electronic components mounted to the rear case 102 are externally exposed.

As illustrated, when the rear cover 103 is coupled to the rear case 102, a side surface of the rear case 102 is partially exposed. In some cases, upon the coupling, the rear case 102 may also be completely shielded by the rear cover 103. In some embodiments, the rear cover 103 may include an opening for externally exposing a camera 121b or an audio output module 152b.

The cases 101, 102, 103 may be formed by injection-molding synthetic resin or may be formed of a metal, for example, stainless steel (STS), aluminum (Al), titanium (Ti), or the like.

As an alternative to the example in which the plurality of cases form an inner space for accommodating components, the mobile terminal 100 may be configured such that one case forms the inner space. In this example, a mobile terminal 100 having a uni-body is formed in such a manner that synthetic resin or metal extends from a side surface to a rear surface.

If desired, the mobile terminal 100 may include a waterproofing unit (not shown) for preventing introduction of water into the terminal body. For example, the waterproofing unit may include a waterproofing member which is located between the window 151a and the front case 101, between the front case 101 and the rear case 102, or between the rear case 102 and the rear cover 103, to hermetically seal an inner space when those cases are coupled.

FIGS. 1B and 1C depict certain components as arranged on the mobile terminal. However, it is to be understood that alternative arrangements are possible and within the teachings of the instant disclosure. Some components may be omitted or rearranged. For example, the first manipulation unit 123a may be located on another surface of the terminal body, and the second audio output module 152b may be located on the side surface of the terminal body.

The display unit 151 outputs information processed in the mobile terminal 100. The display unit 151 may be implemented using one or more suitable display devices. Examples of such suitable display devices include a liquid crystal display (LCD), a thin film transistor-liquid crystal display (TFT-LCD), an organic light emitting diode (OLED), a flexible display, a 3-dimensional (3D) display, an e-ink display, and combinations thereof.

The display unit 151 may be implemented using two display devices, which can implement the same or different display technology. For instance, a plurality of the display units 151 may be arranged on one side, either spaced apart from each other, or these devices may be integrated, or these devices may be arranged on different surfaces.

The display unit 151 may also include a touch sensor which senses a touch input received at the display unit. When a touch is input to the display unit 151, the touch sensor may be configured to sense this touch and the controller 180, for example, may generate a control command or other signal corresponding to the touch. The content which is input in the touching manner may be a text or numerical value, or a menu item which can be indicated or designated in various modes.

The touch sensor may be configured in a form of a film having a touch pattern, disposed between the window 151a and a display on a rear surface of the window 151a, or a metal wire which is patterned directly on the rear surface of the window 151a. Alternatively, the touch sensor may be integrally formed with the display. For example, the touch sensor may be disposed on a substrate of the display or within the display.

The display unit 151 may also form a touch screen together with the touch sensor. Here, the touch screen may serve as the user input unit 123 (see FIG. 1A). Therefore, the touch screen may replace at least some of the functions of the first manipulation unit 123a.

The first audio output module 152a may be implemented in the form of a speaker to output voice audio, alarm sounds, multimedia audio reproduction, and the like.

The window 151a of the display unit 151 will typically include an aperture to permit audio generated by the first audio output module 152a to pass. One alternative is to allow audio to be released along an assembly gap between the structural bodies (for example, a gap between the window 151a and the front case 101). In this case, a hole independently formed to output audio sounds may not be seen or is otherwise hidden in terms of appearance, thereby further simplifying the appearance and manufacturing of the mobile terminal 100.

The optical output module 154 can be configured to output light for indicating an event generation. Examples of such events include a message reception, a call signal reception, a missed call, an alarm, a schedule notice, an email reception, information reception through an application, and the like. When a user has checked a generated event, the controller can control the optical output unit 154 to stop the light output.

The first camera 121a can process image frames such as still or moving images obtained by the image sensor in a capture mode or a video call mode. The processed image frames can then be displayed on the display unit 151 or stored in the memory 170.

The first and second manipulation units 123a and 123b are examples of the user input unit 123, which may be manipulated by a user to provide input to the mobile terminal 100. The first and second manipulation units 123a and 123b may also be commonly referred to as a manipulating portion, and may employ any tactile method that allows the user to perform manipulation such as touch, push, scroll, or the like. The first and second manipulation units 123a and 123b may also employ any non-tactile method that allows the user to perform manipulation such as proximity touch, hovering, or the like.

FIG. 1B illustrates the first manipulation unit 123a as a touch key, but possible alternatives include a mechanical key, a push key, a touch key, and combinations thereof.

Input received at the first and second manipulation units 123a and 123b may be used in various ways. For example, the first manipulation unit 123a may be used by the user to provide an input to a menu, home key, cancel, search, or the like, and the second manipulation unit 123b may be used by the user to provide an input to control a volume level being output from the first or second audio output modules 152a or 152b, to switch to a touch recognition mode of the display unit 151, or the like.

As another example of the user input unit 123, a rear input unit (not shown) may be located on the rear surface of the terminal body. The rear input unit can be manipulated by a user to provide input to the mobile terminal 100. The input may be used in a variety of different ways. For example, the rear input unit may be used by the user to provide an input for power on/off, start, end, scroll, control volume level being output from the first or second audio output modules 152a or 152b, switch to a touch recognition mode of the display unit 151, and the like. The rear input unit may be configured to permit touch input, a push input, or combinations thereof.

The rear input unit may be located to overlap the display unit 151 of the front side in a thickness direction of the terminal body. As one example, the rear input unit may be located on an upper end portion of the rear side of the terminal body such that a user can easily manipulate it using a forefinger when the user grabs the terminal body with one hand. Alternatively, the rear input unit can be positioned at most any location of the rear side of the terminal body.

Embodiments that include the rear input unit may implement some or all of the functionality of the first manipulation unit 123a in the rear input unit. As such, in situations where the first manipulation unit 123a is omitted from the front side, the display unit 151 can have a larger screen.

As a further alternative, the mobile terminal 100 may include a finger scan sensor which scans a user's fingerprint. The controller 180 can then use fingerprint information sensed by the finger scan sensor as part of an authentication procedure. The finger scan sensor may also be installed in the display unit 151 or implemented in the user input unit 123.

The microphone 122 is shown located at an end of the mobile terminal 100, but other locations are possible. If desired, multiple microphones may be implemented, with such an arrangement permitting the receiving of stereo sounds.

The interface unit 160 may serve as a path allowing the mobile terminal 100 to interface with external devices. For example, the interface unit 160 may include one or more of a connection terminal for connecting to another device (for example, an earphone, an external speaker, or the like), a port for near field communication (for example, an Infrared Data Association (IrDA) port, a Bluetooth port, a wireless LAN port, and the like), or a power supply terminal for supplying power to the mobile terminal 100. The interface unit 160 may be implemented in the form of a socket for accommodating an external card, such as Subscriber Identification Module (SIM), User Identity Module (UIM), or a memory card for information storage.

The second camera 121b is shown located at the rear side of the terminal body and includes an image capturing direction that is substantially opposite to the image capturing direction of the first camera unit 121a. If desired, second camera 121a may alternatively be located at other locations, or made to be moveable, in order to have a different image capturing direction from that which is shown.

The second camera 121b can include a plurality of lenses arranged along at least one line. The plurality of lenses may also be arranged in a matrix configuration. The cameras may be referred to as an “array camera.” When the second camera 121b is implemented as an array camera, images may be captured in various manners using the plurality of lenses and images with better qualities.

As shown in FIG. 1C, a flash 124 is shown adjacent to the second camera 121b. When an image of a subject is captured with the camera 121b, the flash 124 may illuminate the subject.

As shown in FIG. 1B, the second audio output module 152b can be located on the terminal body. The second audio output module 152b may implement stereophonic sound functions in conjunction with the first audio output module 152a, and may be also used for implementing a speaker phone mode for call communication.

At least one antenna for wireless communication may be located on the terminal body. The antenna may be installed in the terminal body or formed by the case. For example, an antenna which configures a part of the broadcast receiving module 111 may be retractable into the terminal body. Alternatively, an antenna may be formed using a film attached to an inner surface of the rear cover 103, or a case that includes a conductive material.

A power supply unit 190 for supplying power to the mobile terminal 100 may include a battery 191, which is mounted in the terminal body or detachably coupled to an outside of the terminal body. The battery 191 may receive power via a power source cable connected to the interface unit 160. Also, the battery 191 can be recharged in a wireless manner using a wireless charger. Wireless charging may be implemented by magnetic induction or electromagnetic resonance.

The rear cover 103 is shown coupled to the rear case 102 for shielding the battery 191, to prevent separation of the battery 191, and to protect the battery 191 from an external impact or from foreign material. When the battery 191 is detachable from the terminal body, the rear case 103 may be detachably coupled to the rear case 102.

An accessory for protecting an appearance or assisting or extending the functions of the mobile terminal 100 can also be provided on the mobile terminal 100. As one example of an accessory, a cover or pouch for covering or accommodating at least one surface of the mobile terminal 100 may be provided. The cover or pouch may cooperate with the display unit 151 to extend the function of the mobile terminal 100. Another example of the accessory is a touch pen for assisting or extending a touch input to a touch screen.

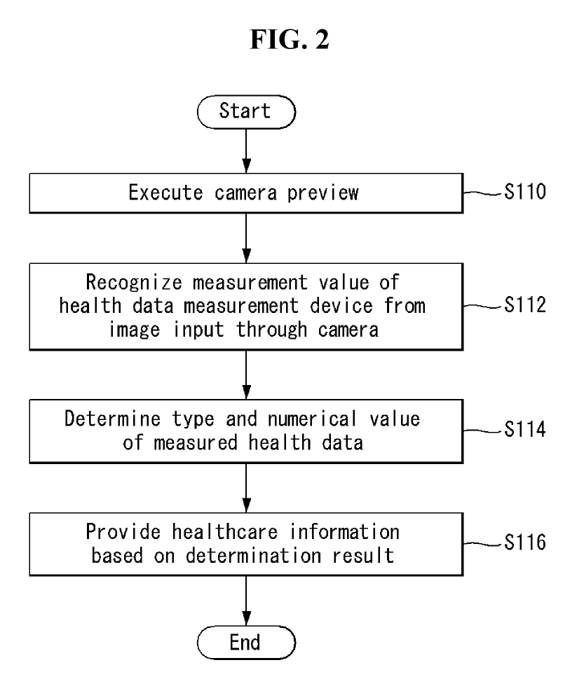

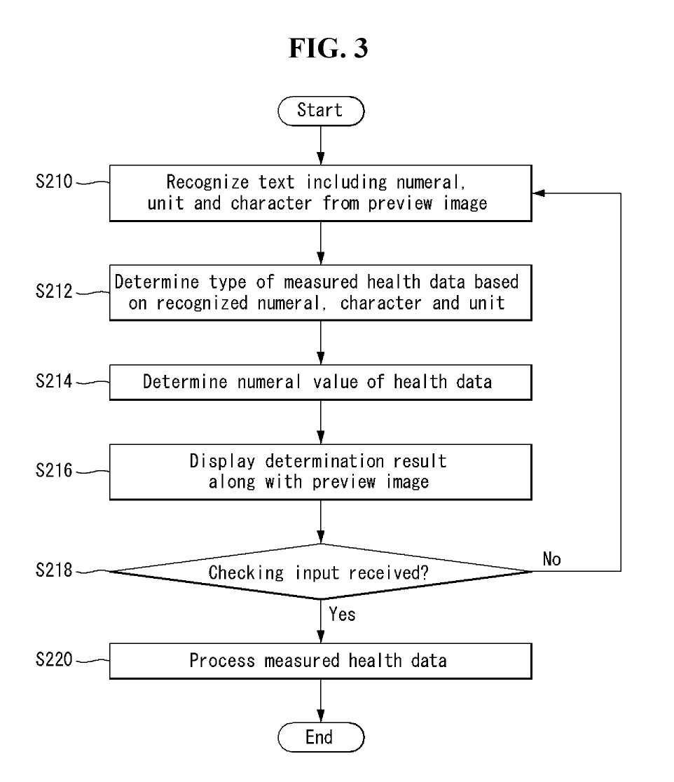

FIGS. 2 and 3 are flowcharts illustrating control of the mobile terminal according to the present invention.

Referring to FIG. 2, the controller 180 of the mobile terminal 100 may display a preview image on the display unit 151 by activating the camera 121 (S110) and recognize a measurement value of a health data measurement device from an image captured by the camera 121 (S112). The health data measurement device may include devices for measuring bio-signals, such as a body weight scale, a manometer, a blood sugar meter, a body composition monitor and a pulse meter, and devices for measuring the intensity of exercise, such as a bicycle speedometer and a treadmill speedometer. The controller 180 of the mobile terminal 200 can receive an image of such health data measurement device through the camera 121 and recognize a measurement value displayed on the device.

The controller 180 may determine the type and numerical value of the measured health data by analyzing the recognized measurement value (S114). For example, the controller 180 can determine which one of weight, blood pressure, blood sugar, body composition and intensity of exercise corresponds to the health data measured based on the numerical range and units of the measurement value.

Subsequently, the controller 180 may provide healthcare information based on the measured health data (S116). The healthcare information may include physical condition determined based on the measured health data, additional information such as solutions and guidelines for healthy living based on the determined physical conditions, and health data measurement history of the user. In addition, the controller 180 may request that the user additionally input other types of health data in order to accurately determine the physical condition of the user. For example, upon determining that the weight of the user exceeds a normal weight, the controller 180 can instruct the user to input a body composition measurement value in order to determine obesity or instruct the user to additionally input health data such as blood pressure, blood sugar level or the like.

The controller 180 may provide a menu for receiving personal information of the user in order to enhance healthcare information reliability. The controller 180 may receive information about the sex, age, height and disease history of the user and medication being taken by the user and store the same in the memory. The controller 180 can provide healthcare information with high reliability on the basis of prestored personal information and input health data.

The controller 180 can guide periodic health data measurement or hospital visits or recommend rules and diet for physical condition improvement upon determining that the physical condition of the user, determined based on the health data, is abnormal. In addition, the controller 180 may restrict display of information that may cause deterioration of the physical condition of the user according to the determined physical condition. For example, the controller 180 can restrict display of advertisements for products that are prohibited for the user, such as alcohol advertisements and snack advertisements upon determining that the user has high blood pressure or diabetes.

An application (referred to as “healthcare application” hereinafter) can be installed in the mobile terminal 100 according to the aforementioned embodiment.

FIG. 3 is a flowchart illustrating control of the mobile terminal 200 according to the present invention to describe the process of recognizing a measurement value of the health data measurement device from an image input through the camera 121.

The controller 180 may recognize a text including numerals, units and characters from a preview image of the health data measurement device, input through the camera 121 (S210). The health data measurement device displays measurement results such as weight, blood pressure, blood sugar, body composition, heart rate, intensity of exercise, etc. The controller 80 can recognize numerals, units, characters and the like from the preview image of the health data measurement device using image recognition.

The controller 180 may determine the type of measured health data on the basis of the recognized numerals, units and characters (S212) and determine the numerical value of the health data (S214). For example, the controller 180 can determine that the measured health data is a weight upon recognition of characters “kg”. If weight input is selected and then the health data measurement device is photographed during initial execution of the health application, the process of determining the type of the measured health data may be omitted.

The controller 180 may display the health data determination result along with the preview image on the display unit 151 (S216). Accordingly, the user can directly check whether the health data has been correctly recognized.

When predetermined user input is received after display of the recognized health data (S218), the controller 180 may provide healthcare information by processing the determined health data (S220).

When user input is not received, the controller 180 can recognize health data from the preview image and display the same.

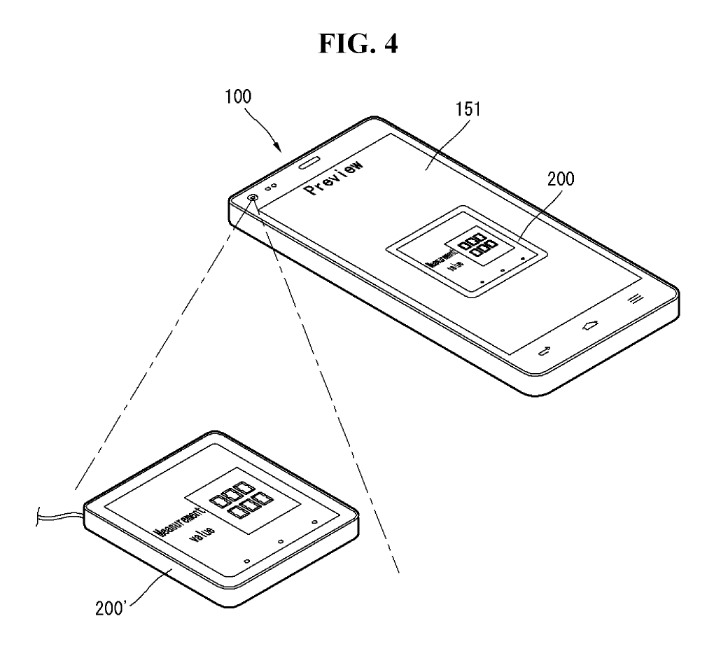

FIGS. 4 to 9 are views illustrating a method of controlling the mobile terminal 100 according to an embodiment of the present invention.

The method of controlling the mobile terminal 100 according to an embodiment of the present invention may be implemented in the environment illustrated in FIG. 4.

Referring to FIG. 4, a health data measurement device 200′ may display a health data measurement result on a display thereof. The health data measurement device 200′ may include devices for measuring bio-signals, such as a body weight scale, a manometer, a blood sugar meter, a body composition monitor and a pulse meter, and devices for measuring the intensity of exercise, such as a bicycle speedometer and a treadmill speedometer.

The controller 180 of the mobile terminal 100 may recognize measurement values from an image of the health data measurement device 200, input through the camera 121. The controller 180 of the mobile terminal 100 displays a preview image on the display unit 151 by activating the camera during execution of the healthcare application. Accordingly, the health data measurement device 200′ can be displayed in the preview image. The controller 180 may recognize measurement values, such as numerals and units, from the image of the health data measurement device 200′ and provide healthcare information on the basis of the recognized measurement values.

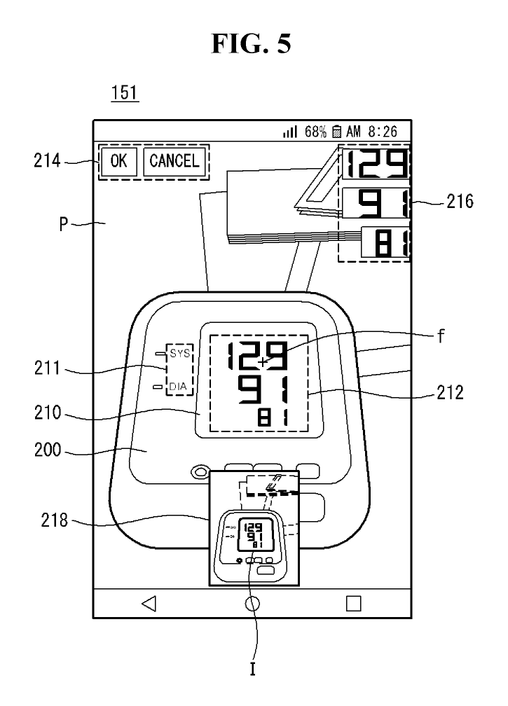

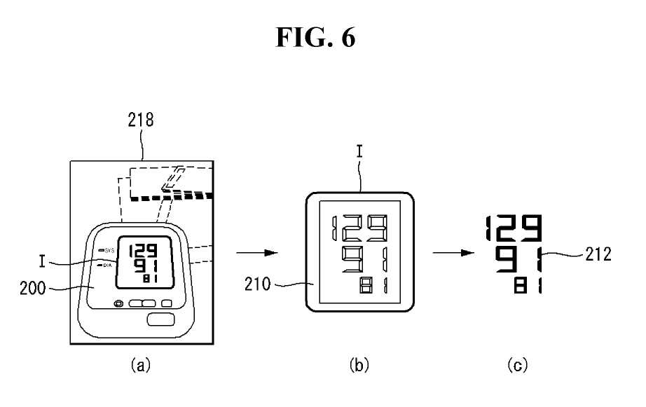

FIGS. 5 and 6 are views illustrating a method of recognizing health data from an image of a health data measurement device 200, captured by the camera 121.

Referring to FIG. 5, the controller 180 of the mobile terminal 100 displays a preview image P on the display unit 151 by activating the camera 121 during execution of the healthcare application. The preview image P may display a focus f thereof and the health data measurement device 200.

The health data measurement device 200 may include a display 210 displaying measurement values. The display 210 can display health data measurement values 212. An indicator 211 for indicating the state of the health data measurement device or providing information related to the health data measurement device 200 may be provided around the display 210.

The controller 180 may recognize the health data measurement values 212 displayed on the display 210 and display the recognition result 216 in a first area of the display unit 151 on which the preview image P is displayed.

The controller 180 may provide an OK/CANCEL menu 214 to the display unit 151 and, when “OK” is selected through the OK/CANCEL menu 214, set the values displayed as the recognized result 216 as the health data measurement values 212. Upon selection of “CANCEL”, the controller 180 re-recognizes the health data measurement values 212.

The controller 180 performs a text recognition process on the area in which the health data measurement values 212 are displayed, in the preview image P, and displays information on a currently recognized area in a second area 218 of the display unit 151.

The second area 218 may display the image used for recognition of the health data measurement values 212 and a recognition region I. To recognize measurement values, the controller 180 can apply various image recognition techniques.

The process through which the controller 180 recognizes the health data measurement values 212 will now be described with reference to FIG. 6.

FIG. 6(a) illustrates the image of the second area 218 displayed on the preview image P. The controller 180 converts resolution of the preview image P into low resolution in order to decrease computational load and then converts the image into an image of a color space that minimizes the influence of light through improved hue, luminance and saturation (IHLS). The controller 180 can display the color space image in the second area 218 and display the recognition region I including numerals to be recognized using a box.

Referring to FIG. 6(b), the controller 180 converts the recognition region I including numerals to be recognized into a standardized size and angle. For example, the controller 180 can use a method of detecting the edge of an image, extracting a contour corresponding to a quadrangle from the edge image and transforming the corresponding region into a smoothed image through homography transformation. Technology for converting the composition, size and angle of a specific region into standardized size and angle can use conventional well-known technology.

Referring to FIG. 6(c), the controller 180 may specify positions of numerals in the standardized region of interest and then compare the specified image with stored images to recognize the health data measurement values 212.

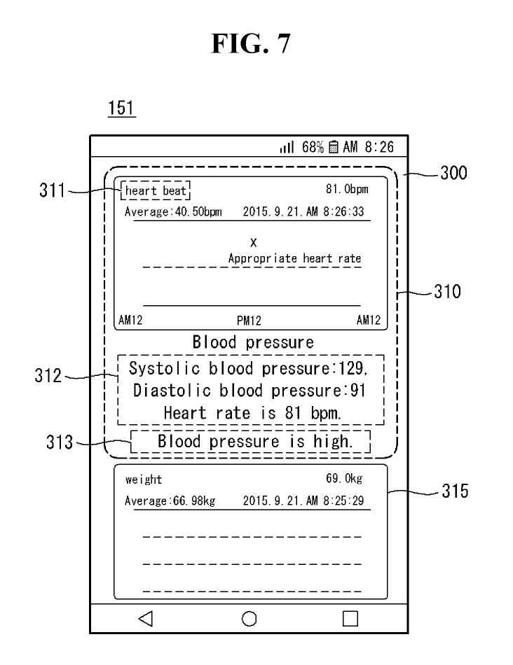

FIG. 7 illustrates a healthcare information screen 300 provided by the mobile terminal 100 according to an embodiment of the present invention. The controller 180 can recognize measurement values of the health data measurement device 200 as an image, determine the type and numerical values of the measurement values and provide healthcare information 310 and 315 based on the measurement values according to determination result.

FIG. 7 illustrates healthcare information 310 displayed when the health data measurement values include blood pressure and heart rate.

When the healthcare information 310 about blood pressure and heart rate is provided, the controller 180 can display measurement value type 311 and measurement result 312 of blood pressure and heart rate. The measurement result 312 may be provided in the form of text, and the measured heart rate may be displayed in the form of a graph along with an appropriate heart rate.

The controller 180 may provide health related information 313 based on the health data measurement value and stored information. For example, a message “Your blood pressure is high” can be displayed as the health related information 313 upon determining that the blood pressure of the user is higher than a reference value.

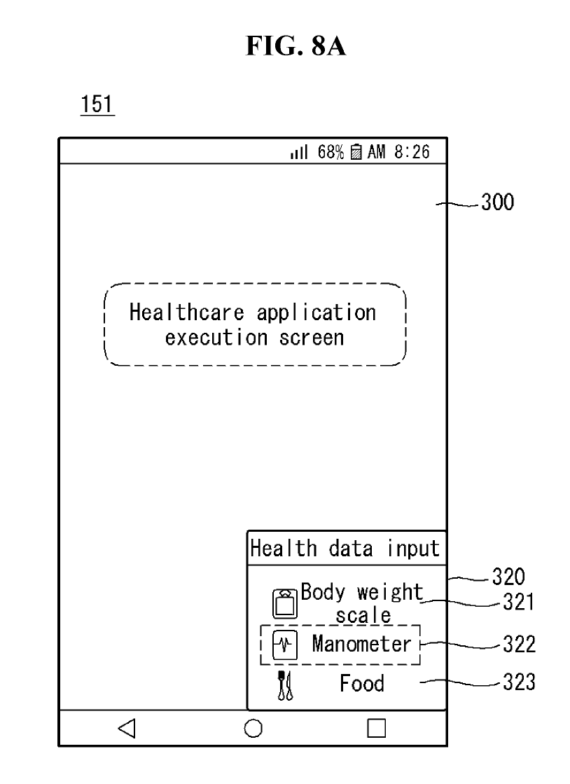

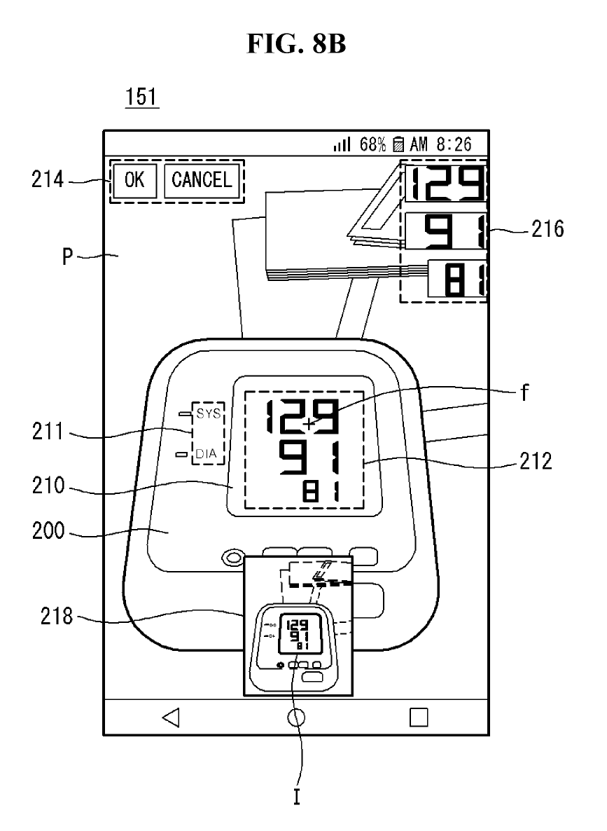

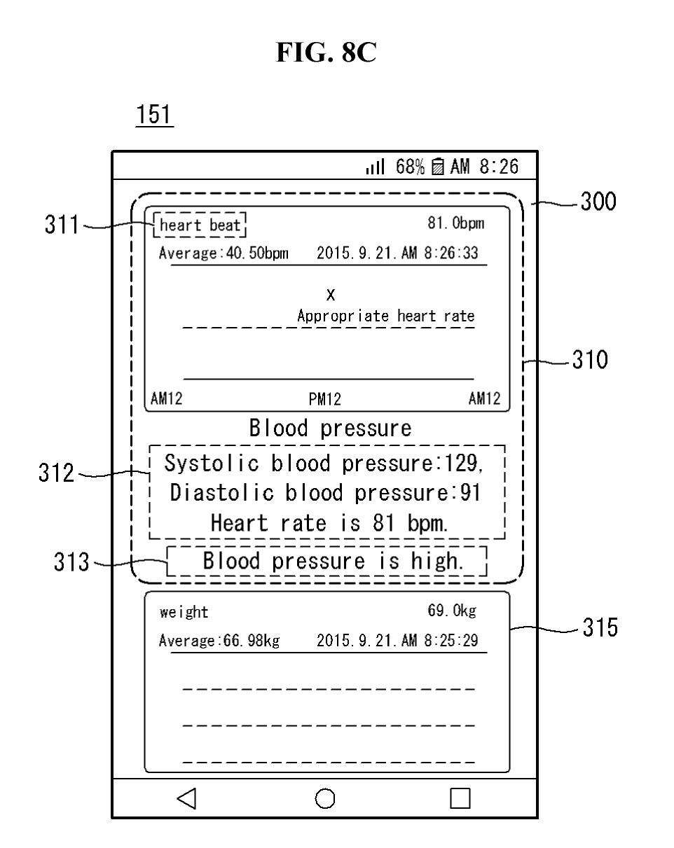

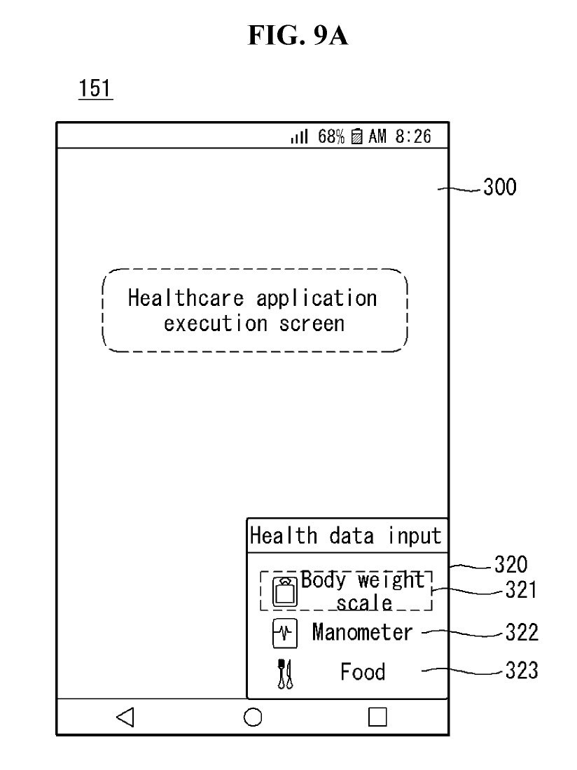

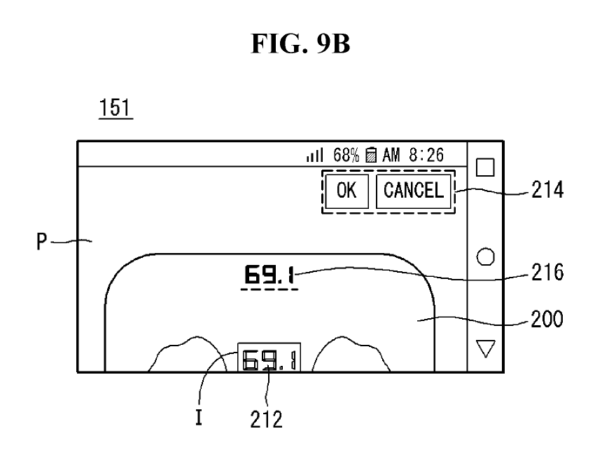

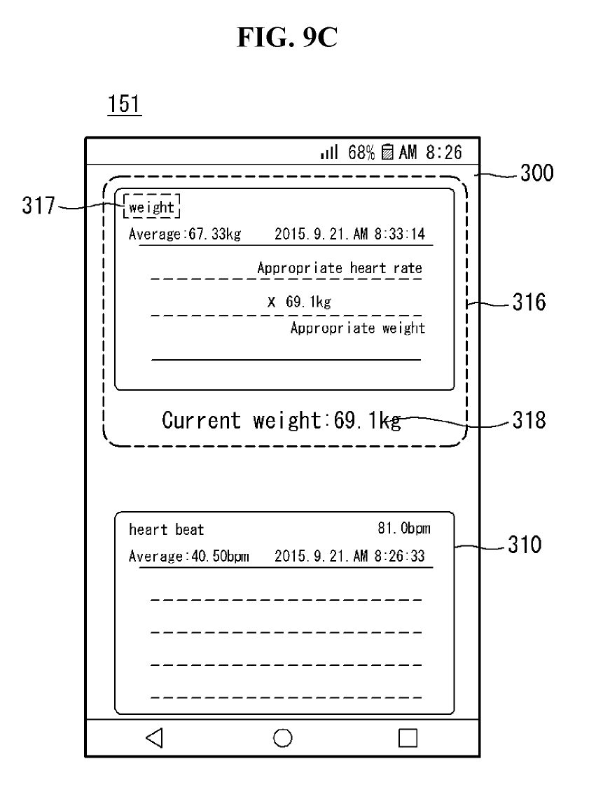

FIGS. 8A and 9A are views illustrating a method of controlling the mobile terminal according to an embodiment of the present invention to describe a process of providing healthcare information by executing the healthcare application that provides healthcare information according to the control method of the present invention.

FIGS. 8A, 8B and 8C illustrate a case in which healthcare information is provided using a manometer as a health data measurement device.

Referring to FIG. 8A, the controller 180 can execute the healthcare application according to user choice and display a health data input menu 320 for selecting the health data measurement device 200.

The health data input menu 320 may include devices for measuring bio-signals, such as a body weight scale 321, a manometer 322, a blood sugar meter, a body composition monitor and a pulse meter, and devices for measuring the intensity of exercise, such as a bicycle speedometer and a treadmill speedometer. The health data input menu 320 may also include a menu through which food 323 eaten by the user is input.

When the manometer 322 is selected through the health data input menu 320, the selected item may be indicated using a box or represented in a color different from other items.

Referring to FIG. 8B, the controller 180 displays the preview image P on the display unit 151 by activating the camera 121 upon selection of the manometer 322 through the health data input menu 320.

The preview image P may display the focus f thereof and the manometer 322 corresponding to the health data measurement device.

The manometer 322 may include a display 210 displaying measurement values. The display 210 may display health data measurement values 212. An indicator 211 for indicating the state of the device may be provided around the display 210.

The controller 180 can recognize the health data measurement values 212 displayed on the display 210 and display the recognition result 216 in the first area of the display unit 151 in which the preview image P is displayed.

The controller 180 can provide the OK/CANCEL menu 214 to the display unit 151 and, when “OK” is selected through the OK/CANCEL menu 214, set the values displayed as the recognition result 216 to the health data measurement values 212. When “CANCEL” is selected, the controller 180 re-recognizes the health data measurement values 212.

The controller 180 can perform text recognition in the area displaying the health data measurement values 121 in the preview image P and display information of the currently recognized region in the second area 218 of the display unit 151. The controller 180 can display the recognition region I for recognizing the health data measurement values 212 in the second area 218. The controller 180 can recognize the health data measurement values using a predetermined image recognition scheme. The controller 180 can hide or display the second area 218 according to user input.

Referring to FIG. 8C, the controller 180 can provide healthcare information 310 on the basis of the measurement values of the manometer 200, recognized using the image recognition scheme.

The manometer 200 can measure blood pressure and heart rate. Accordingly, the controller 180 can display measurement value type 311 and a measurement result 312 of blood pressure and heart rate. The measurement result 312 may be provided as text, and measured heart rate may be displayed in the form of a graph along with an appropriate heart rate.

The controller 180 can provide health related information 313 on the basis of the health data measurement values and stored information. For example, a message “Your blood pressure is high” can be displayed as the health related information 313 upon determining that the blood pressure of the user is higher than a reference value.

The controller 180 can provide a menu for receiving personal information of the user in order to enhance healthcare information reliability. The controller 180 may receive information about the sex, age and height and disease history of the user and medication being taken by the user and store the same in the memory. Accordingly, the controller 180 can provide healthcare information on the basis of prestored personal information and input health data.

FIGS. 9A, 9B and 9C are views illustrating a case in which healthcare information is provided using a body weight scale corresponding to a health data measurement device.

Referring to FIG. 9A, the controller 180 can execute the healthcare application according to user choice and display the health data input menu 320 for selecting the health data measurement device 200.

The health data input menu 320 may include devices for measuring bio-signals, such as a body weight scale 321, a manometer 322, a blood sugar meter, a body composition monitor and a pulse meter, and devices for measuring the intensity of exercise, such as a bicycle speedometer and a treadmill speedometer. The health data input menu 320 may also include the menu through which food 323 eaten by the user is input.

When the body weight scale 321 is selected through the health data input menu 320, the selected item may be indicated using a box or represented in a color different from other items.

Referring to FIG. 9B, the controller 180 displays the preview image P on the display unit 151 by activating the camera 121 upon selection of the body weight scale 321 through the health data input menu 320.

Since the preview image P is displayed to receive a measurement value of the body weight scale 321, an area around the display 210 in which a measurement value is displayed may be photographed. The controller 180 can recognize the measurement value 212 displayed on the display 210 and display the recognition region

The controller 180 can display the recognition result 216 in the first area of the display unit 151 in which the preview image P is displayed and provide the OK/CANCEL menu 214 to the display unit 151. When “OK” is selected through the OK/CANCEL menu 214, the controller 180 can set the value displayed as the recognition result 216 to the health data measurement value 212. When “CANCEL” is selected, the controller 180 re-recognizes the health data measurement value 212.

Referring to FIG. 9C, the controller 180 can provide healthcare information 316 on the basis of the measurement value of the body weight scale 321, recognized using the image recognition scheme.

The body weight scale 321 can measure the weight of the user. Accordingly, the controller 180 can display measurement value type 317 and a weight measurement result 318. The measurement result 318 may be provided as text, and the measured weight may be displayed in the form of a graph along with a standard weight.

The controller 180 can provide a menu for receiving personal information of the user in order to enhance healthcare information reliability. The controller 180 may receive information about the sex, age, height and disease history of the user and medication being taken by the user and store the same in the memory. Accordingly, the controller 180 can provide healthcare information on the basis of prestored personal information and input health data.

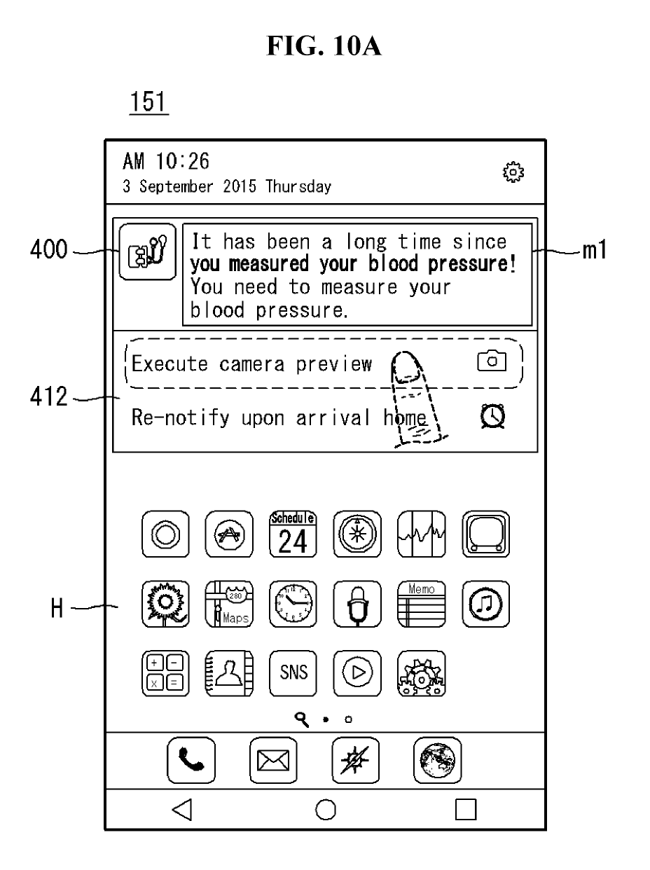

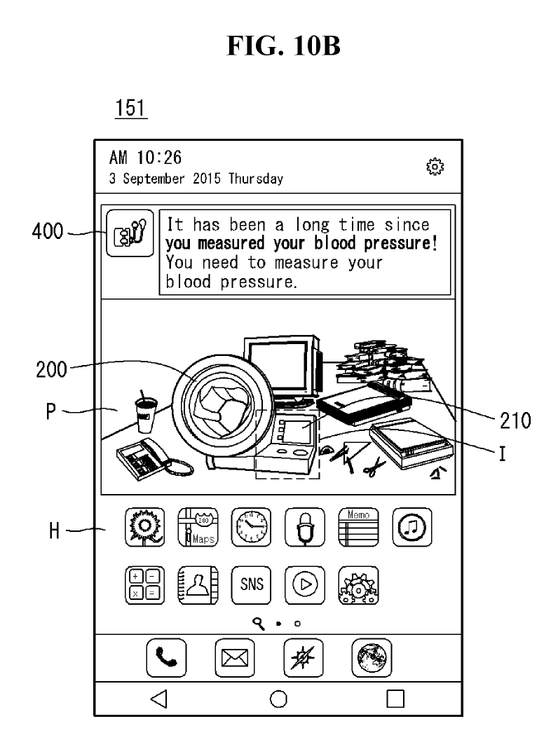

FIGS. 10A to 14 are views illustrating a method of executing a camera preview function for photographing the health data measurement device 200′ in the mobile terminal according to an embodiment of the present invention.

The controller 180 can indicate health data measurement time on the basis of health data input by the user. The controller 180 can display a notification message for instructing the user to periodically measure health data when the health data of the user is not within a normal range. Even when the health data of the user is within the normal range, the controller 180 can display a notification message for instructing the user to measure health data after lapse of a predetermined time from health data measurement.

For example, the controller 180 can display a notification message for instructing the user to measure blood pressure at a specific time everyday upon determining that the user is hypertensive. Even if the blood pressure of the user is normal, the controller 180 can display a notification message for instructing the user to measure blood pressure after a lapse of predetermined time from blood pressure measurement.

FIGS. 10A and 10B illustrate a process of notifying the user of health data measurement after lapse of a reference time from health data measurement and executing camera preview.

Referring to FIG. 10A, the controller 180 can display a message for instructing the user to measure health data through a notification menu 400 upon determining that reference time has elapsed from health data measurement.

The controller 180 displays an indicator region that is provided to a specific region of the home screen H and displays battery charge, network connection, time, a notification message, etc. Upon reception of input of touching the indicator region and dragging down the touch, the controller 180 can display the notification menu 400.

The controller 180 can display a notification message m1 (“It has been a long time since you measured your blood pressure! You need to measure your blood pressure.”) for indicating health data measurement on the notification menu 400, and provide a menu 412 through which a camera review execution command is input.

Upon selection of “camera preview execution” in a menu 412, the selected item can be indicated using a box or represented in a color different from other items.

When “Re-notify upon arrival home” is selected through the menu 412, the controller 180 can indicate health data measurement when the user arrives home. The controller 180 may determine whether the user arrives home using position tracking technology.

Referring to FIG. 10B, upon input of a signal for selecting “camera preview execution” through the menu 412, the controller 180 activates the camera 121 to display the preview image P on the menu 412.

The preview image P may include an image of a blood pressure measurement device 200, which displays a blood pressure measurement value measured by the user. The controller 180 can recognize the display 210 of the blood pressure measurement device 200, which displays the measurement value and is included in the preview image P. The controller 180 can recognize the measurement value displayed on the display 210 and display the recognition region I on the preview image P.

As described above, the controller 180 can display the message m1 for notifying the user of health data measurement in the notification message display area of the home screen H and provide the menu 412 for camera preview execution. Accordingly, the user can check the notification message 400 and then immediately execute camera preview for health data recognition. Therefore, it is possible to omit the process of directly selecting and executing the healthcare application, achieving user convenience.

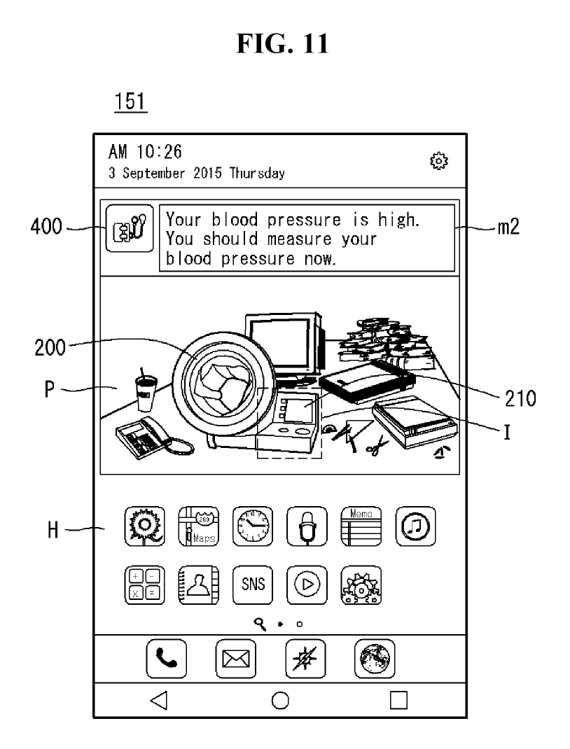

FIG. 11 illustrates a case in which the user is periodically notified of health data measurement. The controller 180 can display a message through the notification menu 400 to instruct the user to periodically measure health data upon determining that health data of the user is not within the normal range.

Referring to FIG. 11, the controller 180 may display a notification message m2 (“Your blood pressure is high. You should measure your blood pressure now”) for indicating health data measurement on the notification menu 400 of the home screen H and, upon reception of predetermined input, activate the camera 121 to display the preview image P on the menu screen 412.

The preview image P can include the image of the blood pressure measurement device 200, which displays a blood pressure measurement value measured by the user. The controller 180 can recognize the display 210 of the blood pressure measurement device 200, which displays the measurement value and is included in the preview image P. The controller 180 can recognize the measurement value displayed on the display 210 and display the recognition region I in the preview image.

Accordingly, the user can check the notification message 400 and then immediately execute camera preview for receiving health data. Therefore, it is possible to omit the process of directly selecting and executing the healthcare application, achieving user convenience.

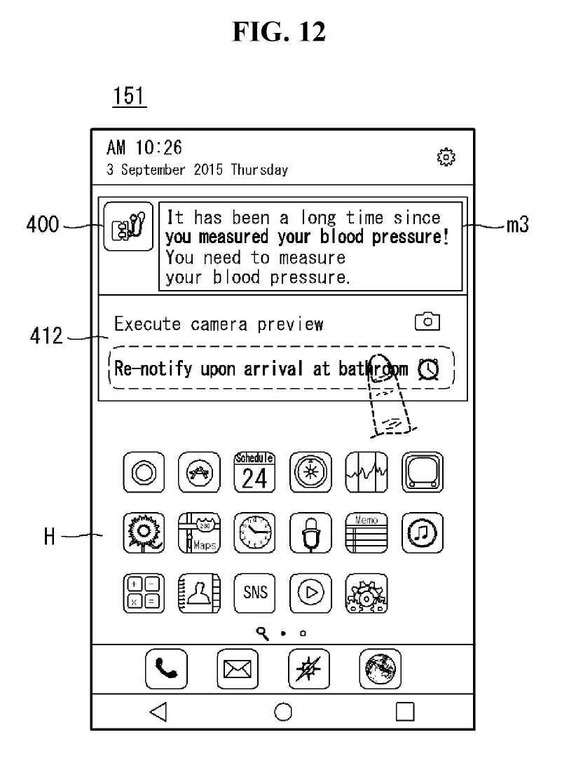

FIG. 12 illustrates an example of providing the menu 412 through which the camera preview execution command is input when a message m3 is displayed through the notification menu 400 to instruct the user to measure health data.

The controller 180 can display the notification message m3 (“It has been a long time since you measured tour blood pressure! You need to measure your blood pressure.”) indicating health data measurement on the notification menu 400 of the home screen H and immediately provide the menu 412 for receiving user choice.

The menu 412 may display “Re-notify upon arrival at bathroom” and “Re-notify upon arrival home” in addition to “camera preview execution”.

The controller 180 can notify the user of health data measurement when the user arrives at a bathroom or arrives home according to a selected item. The controller 180 can determine whether the user arrives at the bathroom or home using a position tracking function. Here, notified places such as the bathroom and home may be previously registered by the user. For example, the user can register a place where the specific health data measurement device 200′ is located such that notification of measurement of corresponding health data can be displayed when the user arrives at the registered place.

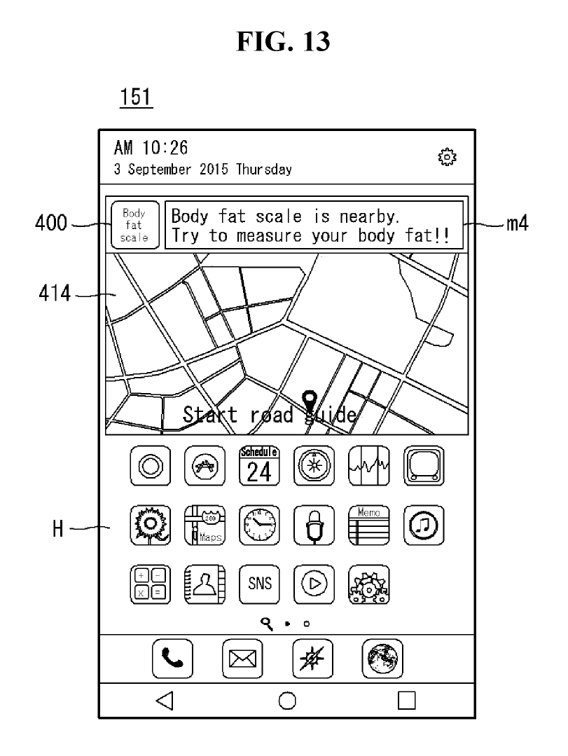

FIG. 13 illustrates an embodiment of displaying a message m4 through the notification menu 400 to instruct the user to measure health data when the user approaches a place where the health data measurement device 200′ is located.

The controller 180 can store information about the place where the health data measurement device 200′ is located, which has been registered by the user, and display the notification menu 400 to instruct the user to measure health data, upon determining that the user approaches the place. The controller 180 may be provided with the information about the place where the health data measurement device 200′ is located from an external server or may enable the user to register the information and store the information.

The controller 180 can display the notification message m4 (“Body fat scale is nearby. Try to check your body fat!”) for notifying the user of health data measurement on the notification menu 400 of the home screen H and provide a map 414 indicating the location.

The map 414 may display a guide menu for guiding a route from the current position to a destination. Upon selection of the guide menu, the controller 180 can guide a route to the location of the health data measurement device 200′.

Accordingly, the user can move to the location of the health data measurement device 200′ and measure health data according to notification of the controller 180 when the user is located in an area close to the health data measurement device 200′.

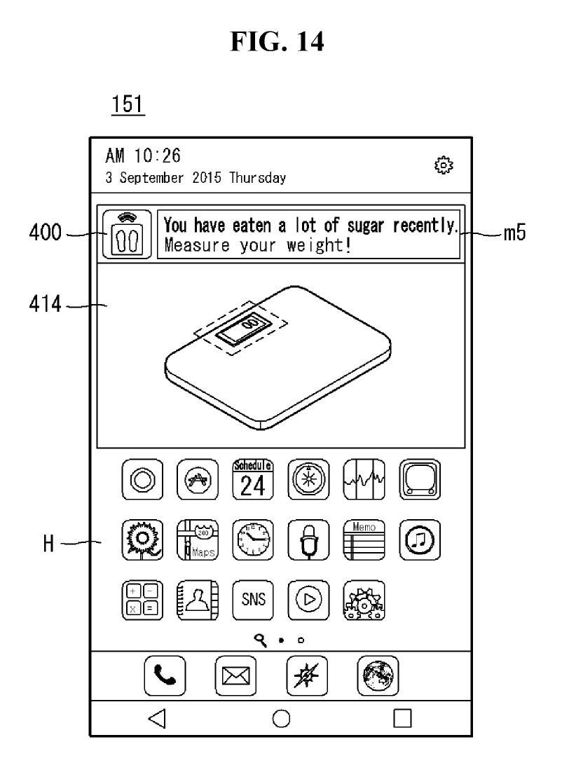

FIG. 14 illustrates an embodiment of displaying a message m5 through the notification menu 400 to instruct the user to measure health data when additional data such as a diet of the user is collected.

The controller 180 can receive and store diet information of the user and display the notification menu 400 such that health data is measured on the basis of the input diet information. The controller 180 can receive and store the diet of the user through the healthcare application or an application related thereto.

The controller 180 can display a notification message m5 (“You have eaten a lot of ‘sugar’ recently. Measure your weight!”) for indicating health data measurement on the notification menu 400 of the home screen H and, upon reception of a predetermined input, activate the camera 121 to display the preview image P on the menu screen 414.

The preview image P can include an image of a body weight scale 200, which displays a weight measurement value measured by the user. The controller 180 can recognize the measurement value of the body weight scale 200, which is included in the preview image P.

Accordingly, the user can check the weight measurement notification message m5 according to the recent diet and then immediately execute camera preview for receiving health data recognition.

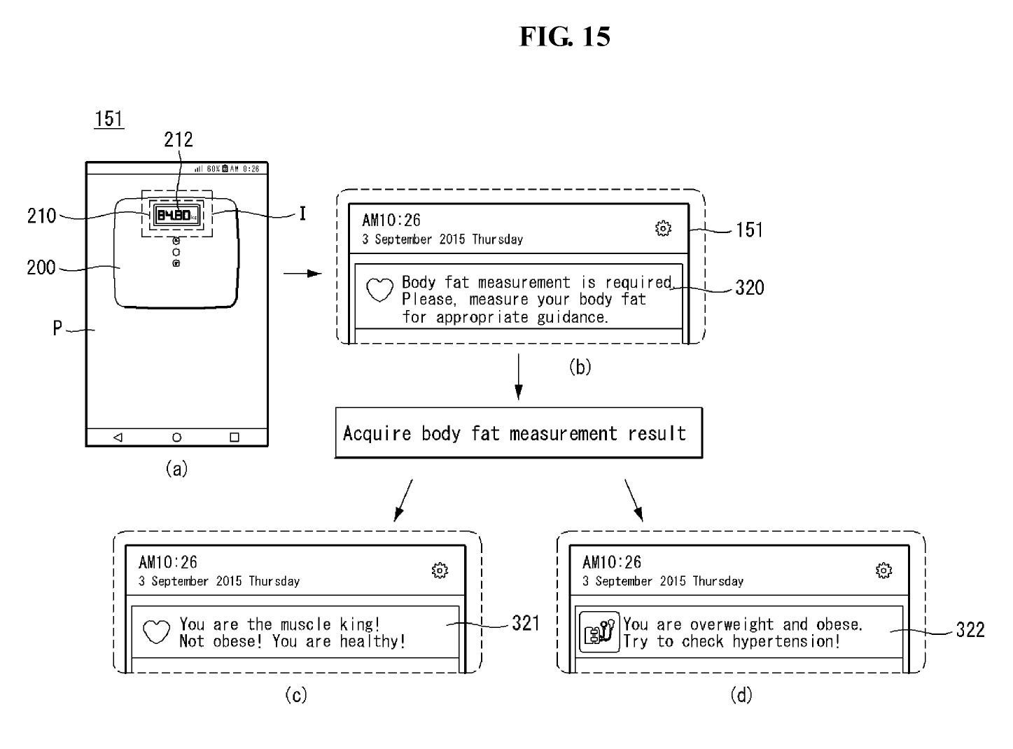

FIGS. 15 to 22 are views illustrating a method of providing healthcare information through the mobile terminal according to an embodiment of the present invention.

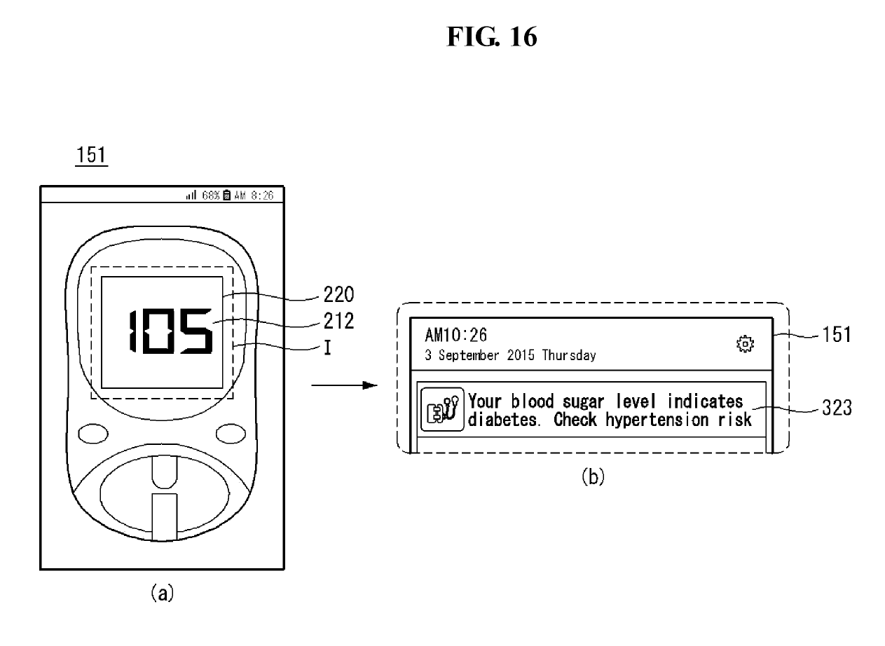

FIGS. 15 and 16 are views illustrating a process of providing healthcare information by requesting additional health data when specific health data is input.

Referring to FIG. 15, the controller 180 activates the camera 121 to display the preview image P including the body weight scale 200 on the display unit 151, as shown in (a). When the preview image P includes the body weight scale 200, the controller 180 can recognize the display 210 of the body weight scale 200, which displays a measurement value 212, and display a recognition region I. The controller 180 can recognize the measurement value 212 displayed on the display 210 to acquire a weight measurement value corresponding to health data. The controller 180 can provide healthcare information such as obesity/normal/underweight on the basis of the weight measurement value.

When it is difficult to provide accurate healthcare information only with weight, the controller 180 may display a message 320 (“Body fat measurement is required. Please, measure your body fat for appropriate guidance.”) for requesting additional health data on the display 151, as shown in (b).

Accordingly, when the user inputs a body fat measurement value, the controller 180 can provide healthcare information based on the weight and the body fat measurement value.

That is, even if the same health data is input, when additionally input health data have different values, different results may be obtained. Accordingly, even if the same weight is input, when different body fat measurement values are additionally input, different results can be provided. For example, when a body fat measurement value is relatively low compared to weight, healthcare information 321 indicating a muscular healthy condition can be provided, as shown in (c). Conversely, when a body fat measurement value is relatively high compared to weight, healthcare information 322 indicating overweight and obesity can be provided, as shown in (d).

Referring to FIG. 16, the controller 180 displays the preview image P including a blood sugar meter on the display unit 151 by activating the camera 121, as shown in (a). When the preview image P includes the blood sugar meter, the controller 180 can recognize a display 220 of the blood sugar meter, which displays a blood sugar measurement value, and display the recognition region I. The controller 180 can recognize the measurement value 212 displayed on the display 220 to acquire the blood sugar measurement value corresponding to health data. The controller 180 can provide healthcare information based on the blood sugar measurement value.

In the case of diabetes, possibility of high blood pressure or obesity increases, in general. That is, when a blood sugar measurement value is not within a normal range, there is high possibility that a blood pressure measurement value and a weight measurement value are also out of normal ranges.

Accordingly, the controller 180 can display a message 323 (“Your blood sugar level indicates diabetes. Check hypertension risk.”) for requesting additional health data, as shown in FIG. 16(b).

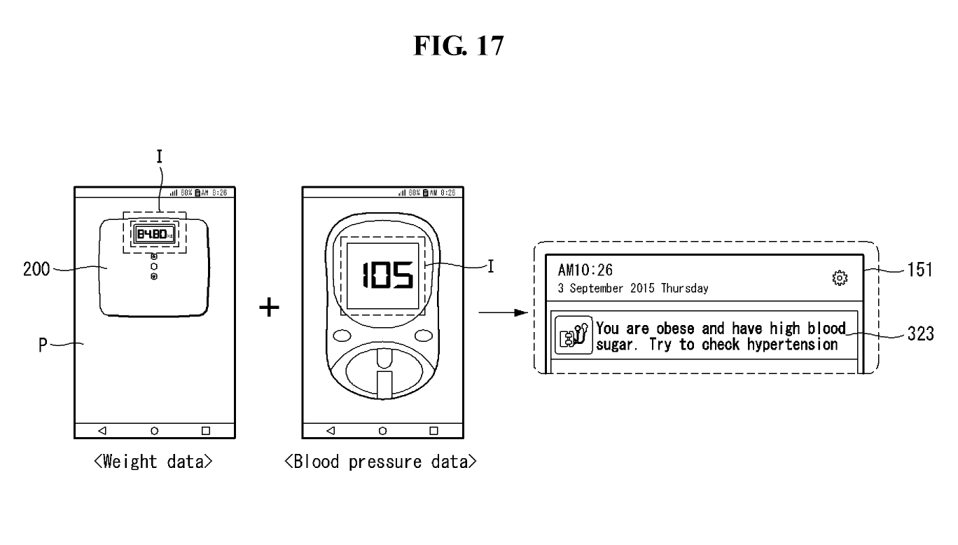

FIG. 17 is a view illustrating a method of providing healthcare information by combining two or more health data.

Referring to FIG. 17, the controller 180 can recognize the display of the body weight scale 200, which displays a weight measurement value, and display the recognition region I when the preview image P includes the body weight scale 200. The controller 180 can recognize the weight measurement value corresponding to health data from the recognition region I.

The controller 180 can recognize the display of the blood sugar meter, which displays a blood sugar measurement value, and display the recognition region I when the preview image P includes the blood sugar meter. The controller 180 can recognize the blood sugar measurement value corresponding to health data from the recognition region I.

Then, the controller 180 can provide healthcare information 323 (“You are obese and have high blood sugar. Check hypertension risk.”) based on the weight measurement value and the blood sugar measurement value.

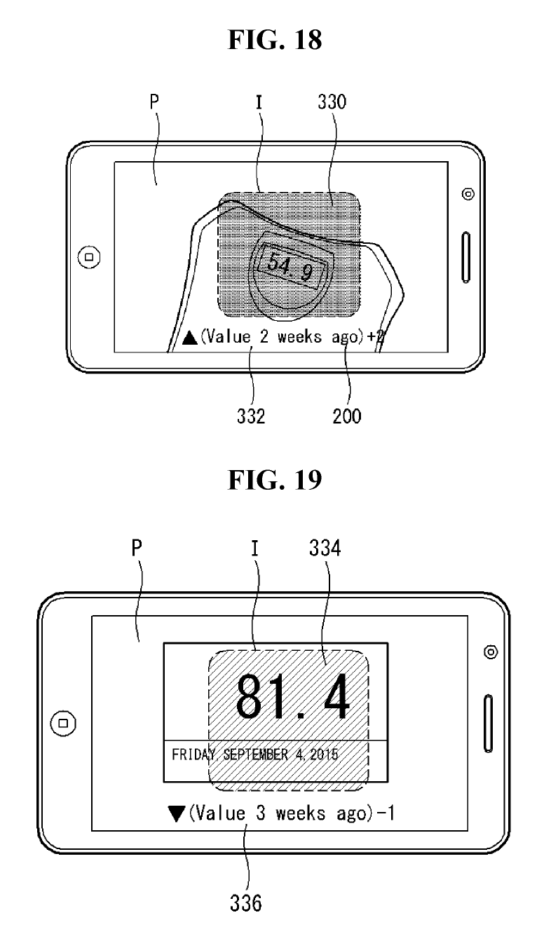

FIGS. 18 and 19 are views for explaining screens providing healthcare information with the preview image P.

Referring to FIG. 18, the controller 180 can recognize the display of the body weight scale 200, which displays a weight measurement value, and display the recognition region I when the preview image P includes the body weight scale 200. The controller 180 can recognize the weight measurement value corresponding to health data from the recognition region I.

The controller 180 can directly display a result 332 obtained by comparing the recognized weight with a stored weight on the preview image P.

The controller 180 can indicate whether the measured weight is within a normal range or out of the normal range using the color of the recognition region I. For example, in the case of a weight within the normal range, the recognition region I can be indicated in green 330.

Referring to FIG. 19, the controller 180 can recognize a display displaying a measurement value in the preview image P and display the recognition region I. The controller 180 can recognize the measurement value from the recognition region I.

The controller 180 can directly display a result 336 obtained by comparing the recognized measurement value with a prestored measurement value on the preview image P.

The controller 180 can indicate whether the measurement result is within a normal range or out of the normal range using the color of the recognition region I. For example, in the case of a measurement result out of the normal range, the recognition region I can be indicated in red 334.

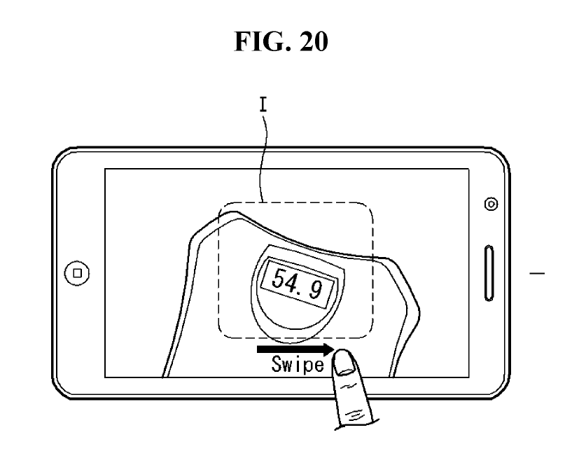

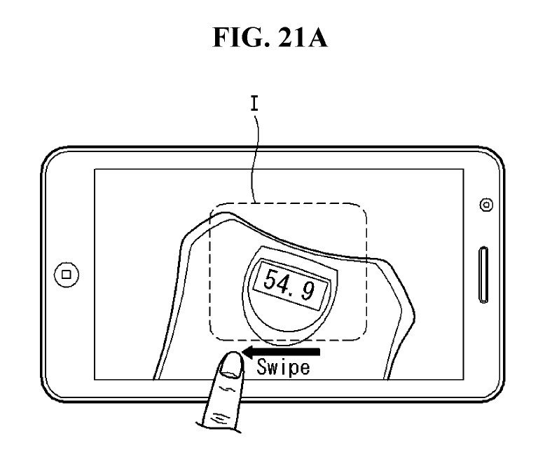

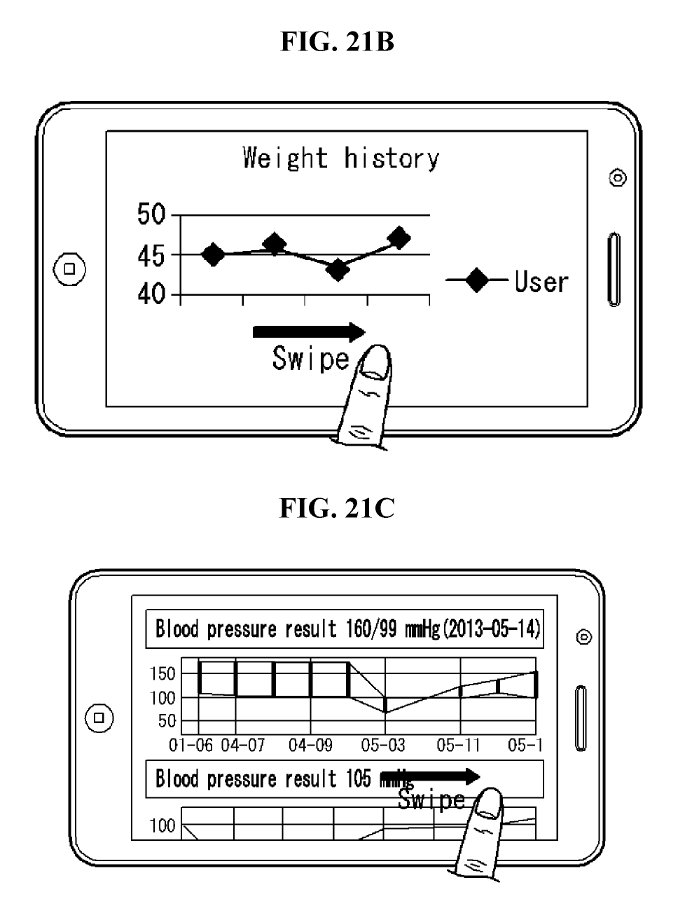

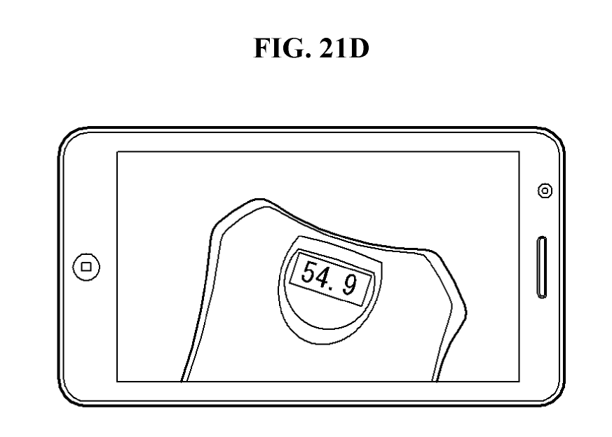

FIGS. 20 and 21D are views for explaining a method of providing healthcare information by processing user input applied through the preview image P.

Referring to FIG. 20, the controller 180 can recognize the display of the body weight scale 200, which displays a weight measurement value, and display the recognition region I when the preview image P includes the body weight scale 200. The controller 180 can recognize the weight measurement value from the recognition region I. When the recognition region I is displayed, the controller 180 can enter a standby mode for receiving user input and fix the currently displayed preview image P on the screen of the display unit 151.

Upon reception of predetermined user input, for example, user input of swiping to the right with the preview image fixed, the controller 180 can enter the preview image display state. In the preview image display state, the controller 180 can perform a process of recognizing the display displaying a measurement value.

FIGS. 21A to 21D are views for explaining a process of receiving user input of swiping to the left with the preview image fixed to provide healthcare data.

Referring to FIG. 21A, the controller 180 can recognize the display of the body weight scale 200, which displays a weight measurement value, and display the recognition region I when the preview image P includes the body weight scale 200. The controller 180 can recognize the weight measurement value corresponding to health data from the recognition region I. When the recognition region I is displayed, the controller 180 can enter a standby mode for receiving user input and fix the currently displayed preview image P on the display unit 151.

Upon reception of predetermined user input, for example, user input of swiping to the left with the preview image fixed, the controller 180 can display history of the currently recognized weight measurement value, as shown in FIG. 21B. Here, when user input of swiping to the right is received, the current state can be switched to the previous state, that is, the state shown in FIG. 21A.

Upon reception of user input of swiping to the left while the history of the currently recognized weight measurement value is displayed as shown in FIG. 21B, the controller 180 can display history of previously stored measurement values, as shown in FIG. 21C. Here, when user input of swiping to the right is received, the current state can be switched to the previous state, that is, the state shown in FIG. 21B.

Upon reception of user input of swiping to the left while the history of previously stored measurement values is displayed as shown in FIG. 21C, the controller 180 can enter the preview image display state, as shown in FIG. 21D. In the preview image display state, the controller 180 can perform a process of recognizing the display displaying a measurement value.

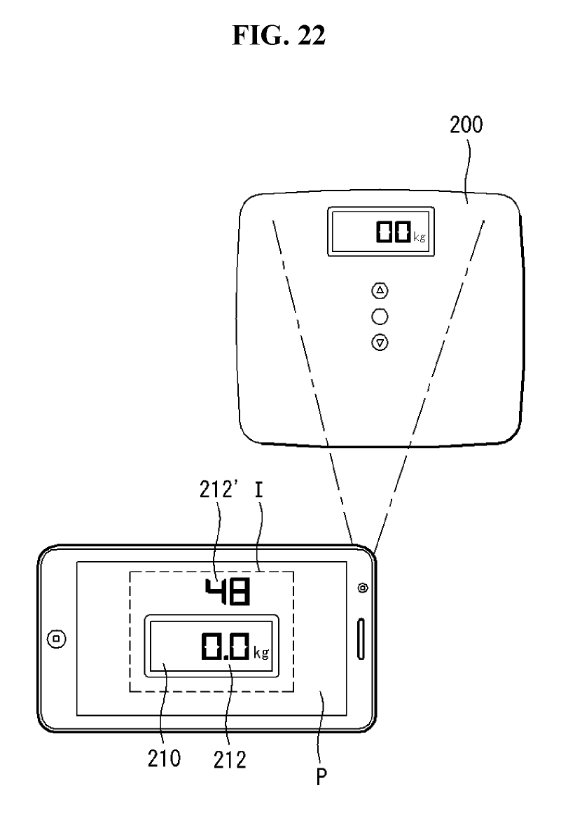

FIG. 22 is a view for explaining another embodiment of providing healthcare information through the preview image P.

Referring to FIG. 22, the controller 180 can recognize the display 210 of the body weight scale 200, which displays a weight measurement value, and display the recognition region I when the preview image P includes the body weight scale 200. The controller 180 can recognize the weight measurement value 212 displayed on the display 210.

When the result of recognition of the weight measurement value 212 indicates that measurement is not performed, for example, when the measurement value is recognized as “0.0”, the controller 180 can determine that the user does not measure the weight. In this case, the controller 180 can display a previously stored weight measurement value 212′ on the preview image P. In this manner, it is possible to immediately check a previous health data measurement value only by photographing an image of the health data measurement device 200.

Various embodiments may be implemented using a machine-readable medium having instructions stored thereon for execution by a processor to perform various methods presented herein. Examples of possible machine-readable mediums include HDD (Hard Disk Drive), SSD (Solid State Disk), SDD (Silicon Disk Drive), ROM, RAM, CD-ROM, a magnetic tape, a floppy disk, an optical data storage device, the other types of storage mediums presented herein, and combinations thereof. If desired, the machine-readable medium may be realized in the form of a carrier wave (for example, a transmission over the Internet). The processor may include the controller 180 of the mobile terminal.

The foregoing embodiments are merely exemplary and are not to be considered as limiting the present disclosure. The present teachings can be readily applied to other types of methods and apparatuses. This description is intended to be illustrative, and not to limit the scope of the claims. Many alternatives, modifications, and variations will be apparent to those skilled in the art. The features, structures, methods, and other characteristics of the exemplary embodiments described herein may be combined in various ways to obtain additional and/or alternative exemplary embodiments.

As the present features may be embodied in several forms without departing from the characteristics thereof, it should also be understood that the above-described embodiments are not limited by any of the details of the foregoing description, unless otherwise specified, but rather should be considered broadly within its scope as defined in the appended claims, and therefore all changes and modifications that fall within the metes and bounds of the claims, or equivalents of such metes and bounds, are therefore intended to be embraced by the appended claims.

Accordingly, an object of the present invention is to address the above-noted and other problems.

Another object of the present invention to provide a mobile terminal capable of interoperating with a health data measurement device even when an additional interface for connecting the health data measurement device to the mobile terminal is not included in the mobile terminal, thereby providing various types of healthcare information, and a method for controlling the same.

According to an aspect of the present invention, a mobile terminal include: a camera; a display; and a controller configured to activate the camera to display a preview image on the display, to recognize a measurement value of a first health data measurement device, included in the preview image, and to provide healthcare information related to the recognized measurement value of first health data.

According to another aspect of the present invention, a mobile terminal includes: a camera; a display; and a controller configured to display a message for requesting measurement of first health data in an indicator region of the display when a predetermined event is generated, to display a preview image on the display by activating the camera upon input of a predetermined signal to the indicator region, to recognize a measurement value of a first health data measurement device, included in the preview image, and to provide healthcare information related to the recognized measurement value of the first health data.

According to another aspect of the present invention, a method for controlling a mobile terminal includes: activating a camera to display a preview image on a display; recognizing a measurement value of a first health data measurement device, included in the preview image; and providing healthcare information related to the recognized measurement value of first health data.

According to another aspect of the present invention, a method for controlling a mobile terminal includes: displaying a message for requesting measurement of first health data in an indicator region of a display when a predetermined event is generated; displaying a preview image on the display by activating a camera upon input of a predetermined signal to the indicator region; recognizing a measurement value of a first health data measurement device, included in the preview image; and providing healthcare information related to the recognized measurement value of the first health data.

The mobile terminal and the method for controlling the same according to the present invention have the following advantages.

According to at least one embodiment of the present invention, it is possible to enable a mobile terminal to interoperate with a health data measurement device even when an additional interface for connecting the health data measurement device to the mobile terminal is not provided by recognizing health data measured from an image of the health data measurement device, photographed using a camera, and providing healthcare information.

According to at least one embodiment of the present invention, it is possible to indicate health data measurement time to a user according to health data of the user and to directly execute a camera preview function for inputting health data when measurement time is indicated, thereby easily performing health data measurement and management.

According to at least one embodiment of the present invention, it is possible to simplify a menu selection process for checking healthcare information by providing various types of healthcare information when predetermined user input is applied to a preview image of a health data measurement device being photographed by the camera, thereby providing user convenience.

Further scope of applicability of the present invention will become apparent from the detailed description given hereinafter. However, it should be understood that the detailed description and specific examples, while indicating preferred embodiments of the invention, are given by illustration only, since various changes and modifications within the spirit and scope of the invention will become apparent to those skilled in the art from the detailed description.

Any reference in this specification to “one embodiment,” “an embodiment,” “example embodiment,” etc., means that a particular feature, structure, or characteristic described in connection with the embodiment is included in at least one embodiment of the invention. The appearances of such phrases in various places in the specification are not necessarily all referring to the same embodiment. Further, when a particular feature, structure, or characteristic is described in connection with any embodiment, it is submitted that it is within the purview of one skilled in the art to affect such feature, structure, or characteristic in connection with other ones of the embodiments.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. A mobile terminal, comprising:
a camera to capture an image;
a display; and
a controller configured to:
receive an input to activate the camera,
in response to activation of the camera, display a preview image on the display, wherein the preview image is obtained by the camera, and the preview image includes an image of a first health data measurement device, and the first health data measurement device includes first health data,
recognize, from the displayed preview image, a first health data measurement value of the first health data measurement device included in the preview image, and
provide healthcare information related to the recognized first health data measurement value.

2. The mobile terminal according to claim 1, wherein the controller is configured to extract information included in the preview image and to recognize type and numerical value of the first health data based on the extracted information, wherein the extracted information includes at least one of a character, a numeral and a symbol included in the preview image.

3. The mobile terminal according to claim 1, wherein the controller is configured to display a menu for selecting a specific health data measurement device and to determine a type of health data based on the selected health data measurement device.

4. The mobile terminal according to claim 1, wherein the controller is configured to display, at a first area of the display, the recognized first health data measurement value, and
upon reception of a predetermined value, the controller is configured to provide healthcare information related to the first health data measurement value.

5. The mobile terminal according to claim 1, wherein the controller is configured to:
compare the first health data measurement value with a stored reference value and determine whether the first health data measurement value is within a prescribed range, and
when the first health data measurement value is determined to not be within the prescribed range, display a message for requesting measurement of second health data related to the first health data.

6. The mobile terminal according to claim 5, wherein the controller is configured to:
activate the camera,
in response to activation of the camera, display another preview image that includes an image of a second health data measurement device, wherein the second health data measurement device includes second health data,
recognize, from the displayed second health data measurement, a second health data measurement value of the second health data measurement device, and
provide the healthcare information based on the first health data measurement value and the second health data measurement value.

7. The mobile terminal according to claim 1, wherein the controller is configured to:
receive a drag input on the display that is displaying the preview image, and
provide healthcare information based on the direction of the drag input.

8. The mobile terminal according to claim 7, wherein the controller is configured to display, on the display, history information of the first health data based on the direction of the drag input.

9. The mobile terminal according to claim 8, wherein the controller is configured to:
receive another drag input on the display while displaying the history information of the first health data, and
display the preview image or history information of a measurement value of second health data based on the direction of the drag input.

10. The mobile terminal according to claim 1, wherein the controller is configured to:
set an area of the preview image to a recognition region and
display, on the display, the recognition region such that the recognition region is discriminated from other areas of the preview image.

11. The mobile terminal according to claim 10, wherein the controller is configured to:
compare the first health data measurement value with a stored reference value and to determine whether the first health data measurement value is within a prescribed range, and
control the recognition region to be displayed in a first manner when the first health data measurement value is determined to be within the prescribed range and the recognition region to be displayed in a second manner when first health data measurement value is determined to not be within the prescribed range, wherein the recognition regions is displayed differently in the second manner than in the first manner.

12. The mobile terminal according to claim 1, wherein the controller is configured to:
compare the first health data measurement value with a previously measured first health data measurement value, and
display, in a second area of the display, a difference between the first health data measurement and the previously measured first health data measurement value.

13. The mobile terminal according to claim 1, wherein the controller is configured to:
compare the first health data measurement value with a stored reference value and to determine whether the first health data measurement value is within a prescribed range, and
periodically display a message for requesting measurement of the first health data when the first health data measurement value is determined to not be within the prescribed range.

14. The mobile terminal according to claim 1, wherein the recognizing the first health data measurement value includes performing an image recognition on a portion of the image of the first health data measurement device.

15. A mobile terminal, comprising:
a camera to capture an image;
a display; and
a controller configured to:
display, in an indicator region of a display, a message for requesting information of first health data when a predetermined event is generated,
receive an input at the indicator region;
in response to the input at the indicator region, display a preview image on the display by activating the camera, wherein the preview image includes an image of a first health data measurement device, and the first health data measurement device includes first health data,
recognize a first health data measurement value of the first health data measurement device included in the preview image, and
provide healthcare information related to the recognized first health data measurement value.

16. The mobile terminal according to claim 15, wherein the controller is configured to display the message for requesting information of the first health data upon determining that the event is generated.

17. The mobile terminal according to claim 15, further comprising a location information device, wherein the controller is configured to:
obtain information of a current location of the mobile terminal by using the location information device,
determine that the current location is a predetermined location,
in response to the determination that the current location is the predetermined location, determine that the event is generated, and
display the message for requesting information of the first health data.

18. The mobile terminal according to claim 15, wherein the recognizing the first health data measurement value includes performing an image recognition on a portion of the image of the first health data measurement device.

19. A method for controlling a mobile terminal, comprising:
activating a camera of the mobile terminal:
in response to activating the camera, displaying a preview image on a display of the mobile terminal, wherein the displayed preview image includes a first health data measurement device, wherein the first health data measurement device;
recognizing a first health data measurement value of the first health data measurement device displayed in the preview image; and
providing healthcare information related to the recognized first health data measurement value

20. The method according to claim 19, further comprising:
displaying a menu for selecting a specific health data measurement device; obtaining health data according to a health data measurement device selected through the menu, and
performing the activating of the camera after the obtaining of the health data.

21. The method according to claim 19, further comprising:
receiving a drag input at the display that is displaying the preview image; and
providing healthcare information set to a direction of the drag input based on the direction of the drag input.

22. The method according to claim 19, further comprising displaying a message for requesting information of the first health data upon determining that an event is generated.

23. The method according to claim 22, further comprising:
obtaining information of a current location of the mobile terminal by using a location information device,
determining that the current location is a predetermined location,
in response to the determination that the current location is the predetermined location, determining that the event is generated, and
displaying the message for requesting information of the first health data.

24. A method for controlling a mobile terminal, comprising:
displaying, in an indicator region of a display, a message for requesting information of first health data when a predetermined event is generated;
receiving an input at the indicator region;
in response to receiving the input at the indicator region, displaying a preview image on the display by activating a camera, wherein the preview image includes an image of a first health data measurement device, wherein the first health data measurement device include first health data;
recognizing a first health data measurement value of the first health data measurement device included in the preview image; and
providing healthcare information related to the recognized first health data measurement value.

25. The method according to claim 24, further comprising displaying a message for requesting information of the first health data upon determining that an event is generated.

26. The method according to claim 25, further comprising:
obtaining information of a current location of the mobile terminal by using a location information device,
determining that the current location is a predetermined location,
in response to the determination that the current location is the predetermined location, determining that the event is generated, and
displaying the message for requesting information of the first health data.
